(19) United States
(12) Patent Application Publication
(10) Pub. No.: US 2019/0080800 A1
(43) Pub. Date: Mar. 14, 2019

(54) METHODS FOR ASSESSING THE POTENTIAL FOR REPRODUCTIVE SUCCESS AND INFORMING TREATMENT THEREFROM

(71) Applicant: Celmatix Inc., New York, NY (US)

(72) Inventor: Piraye Yurttas Beim, New York, NY (US)

(21) Appl. No.: 15/946,488

(22) Filed: Apr. 5, 2018

Related U.S. Application Data
(60) Provisional application No. 62/482,649, filed on Apr. 6, 2017.

(51) Int. Cl. G16H 50/30; G01N 33/569; G01N 33/50; G16H 10/60

(52) U.S. Cl. CPC G16H 50/30; G06F 19/22; G01N 33/56983; G01N 33/56944; G16H 10/60; G01N 33/56938; G01N 33/56927; G01N 33/5005

(57) ABSTRACT

The invention provides methods for analyzing a patient's potential for achieving ongoing pregnancy with respect to a specific fertility treatment. The methods involve obtaining a sample containing microorganisms from an individual, identifying a number of specific microorganisms present in an individual, and comparing these microorganisms to those known to be associated with reproductive success. The individual is then informed of her or his potential reproductive success based upon the results of the comparison.

### CROSS-REFERENCE TO RELATED APPLICATIONS

This application is claims the benefit of and priority to U.S. Provisional Application No. 62/482,649, filed Apr. 6, 2017, the contents of which are incorporated by reference in their entirety.

### BACKGROUND

Approximately one in seven couples has difficulty conceiving. Infertility may be due to a single cause in either partner, or a combination of factors that may prevent a pregnancy from occurring or continuing. Methods of assessing infertility/reproductive success have relied on highly intrusive and/or uncomfortable tests, such as the insertion of an ultrasound wand inside the vagina of an individual (e.g., transvaginal ultrasound), the injection of dye into the cervix and fallopian tubes while laying on a cold imaging table having X-rays taken (e.g., hysterosalpingogram), and/or the insertion of needles into the person's skin to retrieve an often substantial amount of blood, as well as the procurement of semen samples from male counterparts in an uncomfortable examining room in a doctor's office.

Furthermore, even after a couple has undergone these diagnostic procedures, been informed of their prognosis, and subsequently embarks on a treatment protocol based on this prognosis, the outcome may not be in line with the original prognosis. The uncertainty surrounding these prognoses and treatment protocol decisions is a significant challenge for fertility specialists.

Accordingly, there is a need for a method for assessing fertility in a patient that is both accurate and less intrusive.

### SUMMARY

The present disclosure relates to methods and systems for assessing potential reproductive success and informing course of treatment for optimization. Methods and systems of the invention incorporate aspects of a patient's microbiome in making an assessment of the likelihood of reproductive success, recognizing that the presence of certain microorganisms, the overall burden of microorganisms, and/or the diversity of microorganisms have an effect on reproductive ability. Preferably, methods of the invention comprise non-invasive access to a patient's microbiome. Microorganisms are present in an individual's body fluids, such as saliva, nasal secretions, and vaginal secretions and fecal matter. Methods of the invention can be performed on any of those samples, which can be obtained directly or indirectly by non-invasive means.

Analysis of an individual's microbiome to assess potential reproductive success according to the invention provides an assessment that is at least as accurate as those obtained using invasive means. Accordingly, methods of the invention can either be used as the sole means to assessing reproductive success or in conjunction with other forms of assessment.

Generally, methods of the invention comprise obtaining a sample containing microorganisms from an individual, assaying the sample to determine the presence, abundance (e.g., overall microorganism burden), and/or diversity of microorganisms, and comparing the results to a reference set of data having known associations with reproductive success. In some aspects the reference data is determined at different time points across the menstrual or pregnancy cycle in a reference population. Thus, methods of the invention account for fluctuations that may occur within a microorganism profile over time.

In one embodiment, methods of the invention include obtaining a sample, identifying a number of specific microorganisms present in the sample, and comparing these microorganisms to those known to be associated with reproductive success. Once a sample has been obtained, an assay can be conducted to identify a plurality of microorganisms present in the sample. The identified microorganisms are then processed to obtain a subset of microorganisms, which is then compared to a reference set of microorganisms known to be associated with reproductive success. The individual is then informed of her or his potential reproductive success based upon a statistically-significant match between the subset and the reference set.

In one aspect, the sample can be a bodily fluid sample, such as a vaginal secretion, an anal secretion, an oral secretion, or a nasal secretion. In a preferred embodiment, the bodily fluid sample is an oral secretion such as saliva. In another aspect, the microorganisms to be identified from the sample include bacteria and/or viruses.

Microorganisms within the sample can be identified by conducting a sequencing assay on the nucleic acids of the microorganisms. Additionally, or alternatively, assays can involve antibody-based detection of the microorganisms. In one aspect, once the microorganisms are identified, they are then sorted by genus and/or species. In another aspect, the microorganisms suspected of influencing reproductive outcomes are then selected and comprise all or part of the subset of microorganisms. The subset can include, for example, Abiotrophia spp., Achromobacter spp., Acinetobacter spp., Actinobaculum spp., Actinomyces spp., Afipia spp., Aggregatibacter spp., Agrobacterium spp., Alloiococcus spp., Alloscardovia spp., Anaerococcus spp., Anaeroglobus spp., Arcanobacterium spp., Atopobium spp., Bacillus spp., Bacteroides spp., Bacteroidetes spp., Bartonella spp., Bifidobacterium spp., Bordetella spp., Bradyrhizobium spp., Brevundimonas spp., Bulleidia spp., Burkholderia spp., Campylobacter spp., Candida spp., Capnocytophaga spp., Cardiobacterium spp., Catonella spp., Centipeda spp., Chlamydophila spp., Chloroflexi spp., Clostridiales spp., Comamonas spp., Corynebacterium spp., Cronobacter spp., Cryptobacterium spp., Delftia spp., Desulfobulbus spp., Dialister spp., Dolosigranulum spp., Eggerthella spp., Eikenella spp., Enterobacter spp., Enterococcus spp., Erysipelothrix spp., Escherichia spp., Eubacterium spp., Filifactor spp., Finegoldia spp., Fusobacterium spp., Gardnerella spp., Gemella spp., Granulicatella spp., Haemophilus spp., Helicobacter spp., Johnsonella spp., Jonquetella spp., Kingella spp., Klebsiella spp., Kytococcus spp., Lachnospiraceae spp., Lactobacillus spp., Lactococcus spp., Lautropia spp., Leptotrichia spp., Listeria spp., Lysinibacillus spp., Megasphaera spp., Mesorhizobium spp., Methanobrevibacter spp., Microbacterium spp., Mitsuokella spp., Mobiluncus spp., Mogibacterium spp., Moraxella spp., Mycobacterium spp., Mycoplasma spp., Neisseria spp., Ochrobactrum spp., Olsenella spp., Oribacterium spp., Paenibacillus spp., Parascardovia spp., Parvimonas spp., Peptoniphilus spp., Peptostreptococcacea spp., Peptostreptococcus spp., Porphyromonas spp., Prevotella spp., Propionibacterium spp., Proteus spp., Pseudomonas spp., Pseudoramibacter spp., Pyramidobacter spp., Ralstonia spp., Rhodobacter spp., Rothia spp., Sanguibacter spp., Scardovia spp., Selenomonas spp., Shuttleworthia spp., Simonsiella spp., Slackia spp., Solobacterium spp., Staphylococcus spp., Stenotrophomonas spp., Streptococcus spp., Synergistetes spp., Tannerella spp., Treponema spp., Turicella spp., Variovorax spp., Veillonella spp., Yersinia spp.

In accordance with one aspect of the invention, an obtained subset of microorganisms is compared to a reference population of microorganisms known or suspected to affect reproductive outcomes. In one aspect, the reference population includes a set of microorganisms associated with reproductive success. The set includes, for example, Prevotella nigrescens, Aggregatibacter actinomycetemcomitans, Paenibacillus spp., Lactobacillus crispatus, Lactobacillus gasseri, Lactobacillus iners, Lactobacillus jensenii.

In another embodiment, the overall burden of microorganisms is determined for a sample, which is then compared to reference data that includes the overall microbial (microorganism) burden for members of the reference population. In yet another embodiment, the diversity of microorganisms is determined for a sample and then compared to the reference data, which will also include the diversity of microorganisms within members of the reference population.

The results of one or more of these comparisons will inform the course of treatment to be prescribed thereafter. Treatments can include, for example, in vitro fertilization, hormone therapy, and intrauterine insemination (IUI).

In addition to analysis of an individual's microbiome, clinical data and/or genetic data from the individual can also be included in generating the potential probability of reproductive success. Clinical data, such as hormone levels, age, antral follicle count, clinical diagnoses, and Body Mass Index (BMI), can also be obtained from the individual to be used in the generation of the potential for reproductive success. Genetic data, such as mutations in fertility-related genes and gene expression profiles, can be obtained from the patient and used in the generation of the probability for achieving ongoing pregnancy. In one aspect, the clinical and/or genetic data is also compared to data from the reference population, which includes both clinical and genetic data, in order to provide the individual's potential for reproductive success. This reference population can be the same reference population used in the analysis of the individual's microorganisms, or it can be a different reference population.

### BRIEF DESCRIPTION OF DRAWINGS

FIG. 1 depicts female reproduction/fertility related functional biological classifications.

FIG. 2 depicts male reproduction/fertility related functional biological classifications.

FIG. 3 depicts spermatogenic functional biological classifications.

FIG. 4 depicts a diagram of a system of the invention.

FIG. 5 depicts a heatmap of the oral species detected in the samples.

FIG. 6 depicts a heatmap of the one hundred most abundant species detected in the samples.

FIG. 7 depicts the most abundant genera detected the samples.

FIG. 8 depicts a Venn diagram comparing the species with abundance <1% in the samples.

FIG. 9 depicts the composition of the samples at the genus level.

FIG. 10 depicts the functional signatures of the samples.

FIG. 11 depicts the abundance of species associated with positive outcome.

FIG. 12 depicts the abundance of species associated with negative outcome.

### DETAILED DESCRIPTION

The invention relates to methods and systems for assessing potential reproductive success and informing a course of treatment. Methods of the invention use data obtained from the analysis of an individual's microbiome to assess potential reproductive success. In accordance with the present invention, methods involve obtaining a sample containing microorganisms from an individual, assaying the sample to determine the presence, abundance (e.g., overall microorganism burden), and/or diversity of microorganisms in an individual, and comparing these results to a reference set of data having known associations with reproductive success. In some aspects, reference data is determined at different time points across the menstrual or pregnancy cycle of members of the reference population from which the reference data is obtained. In that way, methods of the invention account for fluctuations that occur within the microorganism profile over time.

In addition to the analysis of an individual's microbiome, clinical data and/or genetic data from the individual can also be included in generating the potential probability of reproductive success. Based on the generated potential for reproductive success, a treatment protocol can be recommended.

### Microbiome Data

The human microbiome is comprised of an aggregate of microorganisms that reside within various tissues and body fluids. These microorganisms include bacteria, eukaryotes, and viruses. The presence, abundance, and/or diversity of microorganisms within an individual's microbiome is indicative of the individual's reproductive potential. Methods for identifying and analyzing these microorganisms will be explained in more detail below.

In certain embodiment, the presence of certain genera of bacteria is indicative of the individual's potential for reproductive success. For example, the presence of one genus may indicate a positive or neutral effect on the individual's potential for reproductive success, while another genus may indicate a negative effect on the individual's potential. Exemplary bacterial genera which generally indicate a positive or neutral effect on reproductive success include Prevotella, Aggregatibacter, Paenibacillus, Lactobacillus, Bacteroides, and Fusobacterium.

Exemplary bacterial genera which may indicate a negative effect on reproductive success include Aggregatibacter, Bacteroides, Bergeyella, Burkholderia, Campylobacter, Capnocytophaga, Chlamydia, Eikenella, Enterococcus, Escherichia, Fusobacterium, Gardnerella, Haemophilus, Leptotrichia, Mycoplasma, Neisseria, Peptostreptococcus, Porphyromonas, Prevotella, Sneathia, Streptococcus, Treponema, Tannerella, Trichomonas, and Ureaplasma.

In other embodiments, one or more bacterial species are indicative of the individual's reproductive success. Exemplary bacterial species positively associated with reproductive functioning include, but are not limited to, Prevotella nigrescens, Aggregatibacter actinomycetemcomitans, Lactobacillus crispatus, Lactobacillus gasseri, Lactobacillus iners, and Lactobacillus jensenii. Exemplary bacterial species negatively associated with reproductive functioning include, but are not limited to, for example, Aggregatibacter actinomycetemcomitans, Campylobacter rectus, Chlamydia trachomatis, Eikenella corrodens, Escherichia coli, Fusobacterium nucleatum, Gardnerella vaginalis, Haemophilus influenza, Mycoplasma hominis, Neisseria gonorrhoeae, Porphyromonas gingivalis, Prevotella intermedia, Prevotella nigrescens, Sneathia sanguinegens, Tannerella denticola, Tannerella forsythia, Trichomonas vaginalis, Ureaplasma parvum, and Ureaplasma urealyticum.

Exemplary viruses associated with reproductive functioning include, but are not limited to, human immunodeficiency virus (HIV), cytomegalovirus (CMV), herpes simplex virus (HSV), human papillomavirus (HPV), Adenovirus, Zika virus.

Methods of the invention also include the analysis of eukaryotic microorganisms that can have an effect on reproductive success. One exemplary eukaryotic microorganism includes, but is not limited to, Candida albicans.

In other embodiments, the abundance of microorganisms is indicative of the individual's reproductive success. For example, an individual's overall microbial burden can indicate a positive or negative effect on an individual's potential for reproductive success.

In still other embodiments, the diversity of microorganisms is indicative of the individual's reproductive success. For example, in one aspect, a greater diversity of microorganisms corresponds to a better reproductive outcome, while a lower diversity of microorganisms corresponds to a poorer reproductive outcome.

### Samples

Samples containing microorganisms may be obtained from a variety of sources. Non-limiting examples include the gut, the vagina, the cervix, the respiratory system, the ear, nasal passages, an oral cavity, a sinus, a nostril, the urogenital tract, skin, feces, auditory canal, earwax, breast milk, blood, sputum, urine, saliva, open wounds, secretions from open wounds, and a combination thereof. Surgical means can be used to access internal tissues, such, as, for example, those in the gastrointestinal tract. In one embodiment, the sample can be a bodily fluid sample, such as a vaginal secretion, an anal secretion, an oral secretion, or a nasal secretion. In a preferred embodiment, the bodily fluid sample is an oral secretion, such as saliva.

Samples should be obtained and maintained using procedures that avoid harsh treatments of the samples in order to maintain the composition of the strains of microorganisms as analyzed as much as possible. Factors that should be monitored are, amongst others, temperature, humidity, and contact with air (oxygen). Suitable sampling methods are known to the person of skill, and can be identified by the person of skill without any undue burden.

### Analysis of Microorganisms

Microorganisms of interest can be identified and/or quantified using any one of several methods known in the art, such as, but not limited to, genetic sequencing, culturing, antibody-based detection methods, and quantitative PCR (qPCR).

In one embodiment, methods of the invention involve sequencing of nucleic acids in the sample to identify microorganisms present in the sample. Nucleic acids may be detected generically, without respect to sequence, or may be detected in a sequence-specific manner. Genetic information from the sample can be obtained by nucleic acid extraction from the sample. Methods for extracting nucleic acid from a sample are known in the art. See for example, Maniatis et al., Molecular Cloning: A Laboratory Manual, Cold Spring Harbor, N.Y., pp. 280-281, 1982, the contents of which are incorporated by reference herein in their entirety.

Exemplary sequencing methods include, but are not limited to the following: dideoxy sequencing reactions (Sanger method) using labeled terminators or primers and gel separation in slab or capillary, shotgun sequencing, polymerase chain reaction (PCR), real-time polymerase chain reaction (qPCR), reverse transcription PCR (RT-PCR), multiplex PCR, ligase chain reaction, pyrosequencing, sequencing by synthesis, sequencing by ligation, massively parallel signature sequencing, polony sequencing, SOLiD sequencing, DNA nanoball sequencing, mass spectrometry sequencing, microfluidic sequencing, high-throughput sequencing, Illumina sequencing, HiSeq sequencing, MiSeq sequencing, 16S ribosome sequencing, sequencing by chain termination and gel separation, as described by Sanger et al., PNAS, 74(12): 5463 67 (1977); chemical degradation of nucleic acid fragments. See, Maxam et al., PNAS, 74: 560 564 (1977); sequencing by hybridization. See, e.g., Harris et al., (U.S. patent application number 2009/0156412); Helicos True Single Molecule Sequencing (tSMS). See Harris T. D. et al. (2008) Science 320:106-109; see also, e.g., Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS, 100: 3960-3964 (2003); 454 sequencing (Roche) (Margulies, M et al. 2005, Nature, 437, 376-380); SOLiD technology (Applied Biosystems); Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982); single molecule, real-time (SMRT) technology of Pacific Biosciences; nanopore sequencing (Soni G V and Meller A. (2007) Clin Chem 53: 1996-2001); chemical-sensitive field effect transistor (chemFET) arrays (See e.g., US Patent Application Publication No. 2009/0026082); and use of an electron microscope (Moudrianakis E. N. and Beer M. PNAS USA. 1965 March; 53:564-71), or combinations thereof, incorporated by reference herein.

In a preferred embodiment, the sequencing method is Illumina sequencing, using, for example, Illumina HiSeq or MiSeq sequencers. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection, and identification steps are repeated.

In another preferred embodiment, the method can involve the mapping of the prokaryotic 16S ribosomal RNA (rRNA) gene. 16S rRNA sequencing is a common amplicon sequencing method used to identify and compare microorganisms present within a given sample. 16S rRNA gene sequencing is a well-established method for studying phylogeny and taxonomy of samples from complex microbiomes. The protocol includes the primer pair sequences for the V3 and V4 region that create a single amplicon of approximately ˜460 base pairs (bp). The protocol also includes overhang adapter sequences that must be appended to the primer pair sequences for compatibility with Illumina index and sequencing adapters. The library preparation steps amplify the V3 and V4 region of the 16S rRNA gene using a limited cycle PCR and adds Illumina sequencing adapters and dual-index barcodes to the amplicon target. Up to 96 libraries can be pooled together for sequencing. Sequencing of reads on a MiSeq sequencing machine using paired 300-bp reads can generate 100,000 reads per sample, commonly recognized as sufficient for metagenomic surveys

Sequencing by any of the methods described above and known in the art produces sequence reads. Sequence reads can be analyzed according to any number of methods known in the art to identify the various microorganisms in the sample.

Sequence-specific detection of nucleic acids may also be completed with oligonucleotide probes. An oligonucleotide probe may be capable of hybridizing with a full-length or partial-length gene sequence of interest. In certain aspects, the invention provides a microarray including a plurality of oligonucleotides attached to a substrate at discrete addressable positions, in which at least one of the oligonucleotides hybridizes to a portion of a gene. Methods of constructing microarrays are known in the art. See for example Yeatman et al. (U.S. patent application number 2006/0195269), the content of which is hereby incorporated by reference in its entirety. Moreover, an oligonucleotide probe may be labeled with a detectable tag, such as a fluorescent dye, that may be detected. Alternatively, nucleic acid to be probed may be labeled such that its binding with the oligonucleotide probe is detected (via an attached label). An oligonucleotide probe may be a primer or a longer, different type of oligonucleotide. The oligonucleotide probe may the same type of nucleic acid as the target (e.g., DNA target and DNA oligonucleotide) or the oligonucleotide probe may be a different type of nucleic acid than the target (e.g., DNA target and RNA probe). Non-limiting examples of a label linked to an oligonucleotide probe may be a fluorescent dye, absorbent chemical species, radiolabel, quantum dot, or nanoparticle.

Oligonucleotide probes may also be immobilized on microbeads. Binding of nucleic acids to oligonucleotide probes arranged on microbeads and detection of such nucleic acids is completed in an analogous fashion to that mentioned above for oligonucleotides, such that nucleic acids to-be-analyzed are labeled and their hybridization with an oligonucleotide probe results in the accumulation of detectable signal that can be indirectly interpreted as the presence of a sequence specific region of nucleic acid.

In another embodiment, identification of microorganisms includes the use of antibody-based detection methods. These methods are based on the transformation of a specific biomolecular interaction between antigen and antibody into a macroscopically detectable signal or change in the physical properties of the media. See e.g., Sveshnikov, Peter; “The Potential of Different Biotechnology Methods in BTW Agent Detection: Antibody Based Methods” The Role of Biotechnology in Countering BTW Agents; Vol. 34 of the series NATO Science Series, pp. 69-77 (2001), incorporated herein by reference. Exemplary antibody detection methods include, but are not limited to, enzyme-linked immunoabsorbent assay (ELISA), western blot, immunohistochemistry, immunocytochemistry, flow cytometry and fluorescence-activated cell sorting (FACS), immunoprecipitation, and enzyme linked immunospot (ELISPOT).

In some cases, the detected molecule may be a common structural component of a group of microorganisms common to a taxon (e.g., genus, species, etc.). For example, a protein type or lipid associated with the plasma membrane of a bacterium may be detected. In addition, a secreted molecule, such as a metabolite, may be detected. For example, some bacteria are known to produce short-chain fatty acids such as butyrate, propionate, valerate, and acetate. Thus, secretion of a biochemical marker can be a common characteristic used to sort microorganisms into a given taxon. As another example, a molecule may be a common metabolite produced by microorganisms within a given taxon, which can also be used to identify and sort microorganisms into taxa. Furthermore, detection of one or more molecules in combination may be used to enumerate a microbial taxon. Other identification methods include spectroscopic methods, such as, but not limited to, optical methods (e.g., UV-Vis absorbance, fluorescence, bioluminescence, Fourier-transform infrared (FT-IR) spectroscopy), nuclear magnetic resonance (NMR) spectroscopy, dynamic light scattering, and mass spectrometry.

Moreover, nucleic acids may be downstream molecules synthesized as the result of gene transcription and/or metagenomic molecules present in a microorganism. For example, in the case of the 16S rRNA gene, genomic DNA corresponding, in whole or part, to regions of the 16S rRNA gene, messenger RNA (mRNA) transcripts, in whole or part, of the 16S rRNA gene, and/or functional 16S rRNA may be detected and used to enumerate the abundance of a microbial taxon characterized by sequence homology of a particular 16S rRNA gene sequence.

Identification of microorganisms and sorting of them into taxa may also be achieved by other means such as analyzing proteomes, transcriptomes, metabolomes, or combinations thereof. For example, microbial RNA transcripts, proteins, non-16S genes, etc. may be profiled.

In accordance with certain aspects, methods of the invention involve the identification of about 1 to about 1,000 microorganisms, for example, 1, 2, 3, 4, 5, 6, 7, 8, 9, 10, 15, 20, 25, 30, 35, 40, 45, 50, 60, 70, 80, 100, 120, 140, 160, 180, 200, 500, or more microorganisms, and any integer therebetween, from a sample of an individual (e.g., a patient).

In some embodiments, the abundance of individual microorganisms is determined. In other embodiments, the overall microbial (or microorganism) burden is determined. Quantitative PCR (qPCR, or real-time PCR) can be conducted to provide an accurate and sensitive method for quantification of individual species and microbial populations as well as the overall microbial burden of a sample. In qPCR, fluorescent dyes are used to label PCR products during thermal cycling. The accumulation of fluorescent signal during the exponential phase of the reaction is measured in order to quantify the PCR products. See e.g., Ott et al., J. Clin. Microbiol., 2004; 42(6); 2566-2572; and Fey et al., Appl. Environ. Microbiol. 2004; 70(6): 3618-3623; and Lyons et al., J Clin Microbiol.; 2000; 38(6): 2362-5. When determining overall microbial burden, qPCR can be used to measure the ratio of microbial to human DNA by, for example, quantifying eukaryotic versus prokaryotic ribosomal RNA.

Any number of methods, both qualitative and quantitative, can be used to further analyze the effect of an individual's microorganism makeup on the potential for reproductive success.

In one aspect, the processing of identified microorganisms involves the sorting the microorganisms by genus and/or species. For example, certain genus may contribute positively to an individual's potential for reproductive success, while others may negatively affect the potential. This can be done by referencing one or more databases and/or other relevant sources, in which the identified microorganisms have already been sorted into various taxa (e.g., genus, species, etc.). Exemplary taxonomy data can be found in, for example, Bergey's Manual of Systematic Bacteriology; the Human Oral Microbiome Database (HOMD), http://www.homd.org/, an online curated set of microbiome species specific to the human oral region; the International Journal of Systematic and Evolutionary Microbiology (IJSB/IJSEM), which includes bacterial and archaeal taxonomy; and www.taxonomicoutline.org/, an online taxonomic outline of available bacteria and archaea.

In one embodiment, once sorted, a subset of microorganisms can be obtained for further analysis. For example, microorganism species within the genera Prevotella, Porphyromonas, Actinomyces, Veillonella, Haemophilus, Streptococcus, Rothia, Fusobacterium, Campylobacter, Selenomonas, Eubacterium, Oribacterium, Bradyrhizobium, Granulicatella, Candida, Capnocytophaga, Bacteroidetes, Atopobium, Lachnospiraceae, Paenibacillus, Solobacterium, Propionibacterium, Gemella, Lautropia, Megasphaera, Kingella, Tannerella, Leptotrichia, and Neisseria that were identified from the sample may be included in the subset. In one aspect, the subset can be about 10, 20, 30, 40, 50, 60, 70, 80, 90, 95 percent, and any percentage in-between, of the initially identified microorganisms. In a preferred embodiment, the subset includes one or more of the following microorganisms: Prevotella, Porphyromonas, Actinomyces, Veillonella, Haemophilus, Streptococcus, Rothia, and Fusobacterium. It is also to be understood that a subset of microorganisms need not be obtained; the analysis can proceed using all of the identified microorganisms.

In accordance with one aspect, the obtained subset (or all of the identified microorganisms) is compared to a reference population of microorganisms known or suspected to affect reproductive outcomes. In one aspect, the reference population includes a set of microorganisms associated with reproductive success. The set includes, for example Prevotella nigrescens, Aggregatibacter actinomycetemcomitans, Paenibacillus spp., Lactobacillus crispatus, Lactobacillus gasseri, Lactobacillus iners, and Lactobacillus jensenii. The reference population can be determined from subjects, such as a cohort of patients, for which pregnancy and fertility outcomes are known.

Methods for assessing an individual's potential for reproductive success generally involve the determination of one or more correlations between the presence, abundance (such as the overall microorganism burden), and/or diversity of microorganisms, and known pregnancy and infertility-related outcomes from a reference set of data to provide a model representative of the potential for reproductive success. The model can then be applied to the input data to generate the potential for reproductive success in the individual, or patient, which will in turn, inform the course of treatment for the patient.

In certain embodiments, the subset is compared to the reference set of microorganisms. In one aspect, the reference set of microorganisms all positively contribute to the individual's potential for reproductive success. Thus, the higher the number of matches between the subset and the reference set, the greater the individual's potential for reproductive success. Preferably, the comparison results in a statistically significant match between the subset and the reference set. In another aspect, the reference set of microorganisms negatively contribute to the individual's potential for reproductive success. Thus, the higher the number of matches between the subset and the reference set, the lower the individual's potential for reproductive success, and vice versa.

Additionally or alternatively, the overall microbial burden of the individual can be compared to the overall microbial burdens determined from the reference data to provide an indication as to the individual's potential for reproductive success (e.g., a higher overall burden may be positively correlated with reproductive success, while a lower overall burden is negatively associated with reproductive success, or vice versa). For example, the reference data can be used to develop a scale of correlation with reproductive success, such that the overall microbial burden of the individual can be compared to the scale in order to provide an indication of the individual's potential for reproductive success. Similar to a scale, a scoring system can also be used, wherein a higher score indicates a better reproductive outcome and a lower score indicates a worse reproductive outcome, or vice versa. In another example, the reference data can be used to determine threshold burden values associated with different levels of reproductive success, such that the overall burden of the individual can be compared to the threshold values in order to provide an indication of the individual's potential for reproductive success.

In another embodiment, the diversity of microorganisms within a sample can be compared to the reference data to provide an indication of the individual's potential for reproductive success (e.g., a greater diversity within the sample can correlate to a positive reproductive outcome, while a lower diversity can correlate to a negative reproductive outcome). Similar to microbial burden, this can be implemented using, for example, any one of a diversity scale, score, or threshold value system.

It is to be understood that any or all of the above-described methods with respect to the presence, abundance, overall burden, and diversity, can be conducted separately or combined to provide an individual's potential for reproductive success.

In yet other embodiments, the microorganism data obtained from the reference population can be passed through an association analysis in order to determine whether and to what extent the presence, abundance, and/or diversity of microorganisms identified within the subjects in the reference population are associated with the potential for reproductive success.

The association analysis involves the use of any one of a number of models to calculate the potential for reproductive success for the reference population, such as a cohort of patients. In certain embodiments, the model also incorporates and adjusts for clinical and/or genetic information, both of which are discussed in more detail below. In one aspect, the model can be weighted towards more recent data.

Suitable analysis methods include, without limitation, logistic regression, ordinal logistic regression, linear or quadratic discriminant analysis, clustering, principal component analysis, nearest neighbor classifier analysis, and discrete time-proportional hazards models.

Logistic regression analysis may be used to generate an odds ratio and relative risk for each characteristic. Method of logistic regression are described, for example in, Ruczinski (Journal of Computational and Graphical Statistics 12:475-512, 2003); Agresti (An Introduction to Categorical Data Analysis, John Wiley & Sons, Inc., 1996, New York, Chapter 8); and Yeatman et al. (U.S. patent application number 2006/0195269), the content of each of which is hereby incorporated by reference in its entirety.

Some embodiments of the present invention provide generalizations of the logistic regression model that handle multicategory (polychotomous) responses. Such embodiments can be used to discriminate an organism into one or more prognosis groups with respect to reproductive success (e.g., good prognosis, poor prognosis). Such regression models use multicategory logit models that simultaneously refer to all pairs of categories, and describe the odds of response in one category instead of another. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference.

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In one embodiment, the selected microorganisms serve as the requisite continuous independent variables. The prognosis group classification of each of the members of the reference population serves as the dichotomous categorical dependent variable. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, New York, incorporated herein by reference.

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

In some embodiments of the present invention, decision trees are used to classify patients. Decision tree algorithms belong to the class of supervised learning algorithms. The aim of a decision tree is to induce a classifier (a tree) from real-world example data. This tree can be used to classify unseen examples which have not been used to derive the decision tree. In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, but are not limited to classification and regression trees (CART), multivariate decision trees, ID3, and C4.5.

In some embodiments, the microorganism data are used to cluster a training set. Additional information and examples are described in Duda and Hart, Pattern Classification and Scene Analysis, 1973, John Wiley & Sons, Inc., New York; Kaufman and Rousseeuw, 1990, Finding Groups in Data: An Introduction to Cluster Analysis, Wiley, New York, N.Y.; Duda, Pattern Classification, Second Edition, 2001, John Wiley & Sons, Inc; and Hastie, 2001, The Elements of Statistical Learning, Springer, New York; Everitt, 1993, Cluster analysis (3rd ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Other algorithms for analyzing associations are known. For example, the stochastic gradient boosting is used to generate multiple additive regression tree (MART) models to predict a range of outcome probabilities. A different approach called the generalized linear model, expresses the outcome as a weighted sum of functions of the predictor variables. The weights are calculated based on least squares or Bayesian methods to minimize the prediction error on the training set. A predictor's weight reveals the effect of changing that predictor, while holding the others constant, on the outcome. In cases where one or more predictors are highly correlated, in a phenomenon known as collinearity, the relative values of their weights are less meaningful; steps must be taken to remove that collinearity, such as by excluding the nearly redundant variables from the model. Thus, when properly interpreted, the weights express the relative importance of the predictors. Less general formulations of the generalized linear model include linear regression, multiple regression, and multifactor logistic regression models, and are highly used in the medical community as clinical predictors.

In another embodiment, a hierarchical clustering of the abundance of species across samples is carried out. Hierarchical Clustering Analysis (HCA) allows us to build clusters of similarly abundant species in a sample population. This is achieved by use of a distance measure between pairs of observations (manhattan, euclidean, maximum), and a linkage criterion (complete, single, mean, Ward's) which specifies the dissimilarity of sets as a function of the pairwise distances of observations in the sets. Hierarchical clustering is used to determine similarly abundant subsets of species, both within and across samples. Such clustering of species populations based on abundance levels provides a method to characterize signatures for individual samples, creating a mechanism to differentiate between samples.

In yet another embodiment, a discrete time-proportional odds model, such as the Cox proportional hazards model, is used to determine the potential for reproductive success in a group of subjects. See e.g., Cox, David R (1972). “Regression Models and Life-Tables”. Journal of the Royal Statistical Society, Series B. 34 (2): 187-220, incorporated herein by reference. Proportional hazards models relate the time that passes before some event occurs to one or more covariates that may be associated with that quantity of time, wherein the unique effect of a unit increase in a covariate is multiplicative with respect to the hazard rate (e.g., odds of achieving reproductive success).

Once the model has been developed based on the reference set of information, the model can then be applied to the microbiome data obtained from the patient to provide the patient's potential for reproductive success. In one aspect, the potential can be provided for any number of fertility treatments in the event that fertility treatments and outcomes are known in the reference population. This information will then inform course of treatment for the individual. In another aspect, the model is dynamic, taking into account any fluctuations in the presence, abundance, overall burden, and/or diversity of microorganisms that occur over the course of a menstrual cycle or over the course of a pregnancy in the reference population. In this way, methods of the present invention are able to provide an individual's potential for reproductive success at a selected point in time using a particular fertility treatment.

Clinical and/or Genetic Data

In addition to analysis of an individual's microbiome, genetic data and/or clinical data from the individual can also be included in generating the potential for reproductive success. In one aspect, the genetic and/or clinical data are also compared to data from the reference population, which includes both clinical and genetic data, in order to provide the individual's potential for reproductive success. As with the microbial data, the clinical and genetic data can be obtained at various points along the menstrual or pregnancy cycle in order to provide a dynamic model. The reference population can be the same reference population used in the analysis of the individual's microorganisms, or it can be a different reference population.

i. Clinical Data

Assessment and analysis of the potential for achieving ongoing pregnancy and live birth incorporates the use of clinical fertility-associated information, or data, such as phenotypic and/or environmental characteristics. Exemplary clinical information is provided in Table 1 below.

TABLE 1
Clinical Information

Cholesterol levels on different days of the menstrual cycle
Age of onset of menses (menarche) for patient and female blood relatives (e.g., sisters, mother,
grandmothers)
Age of menopause for female blood relatives (e.g., sisters, mother, grandmothers)
Number of previous pregnancies (biochemical/ectopic/clinical/fetal heart beat detected, live
birth outcomes), age at the time, and outcome for patient and female blood relatives (e.g.,
sisters, mother, grandmothers)
Diagnosis of Polycystic Ovary Syndrome (PCOS)
Basal Antral Follicle Count (bAFC)
Number of embryos transferred
Pre-implantation Genetic Screening (PGS) results
History of hydrosalpinx or tubal occlusion
History of endometriosis, pelvic pain, or painful periods
Cancer history/type of cancer/treatment/outcome for patient and female blood relatives (e.g.,
sisters, mother, grandmothers)
Age that sexual activity began, current level of sexual activity
Smoking history for patient and blood relatives
Travel schedule/number of flying hours a year/time difference changes of more than 3 hours
(Jetlag and Flight-associated Radiation Exposure)
Nature of periods (duration of menses, duration of cycle)
Biological age (number of years since first menses)
Birth control use
Drug use (illegal or legal)
Body mass index (BMI; current, lowest ever, highest ever)
History of polyps (e.g., uterine, endometrial)
History of hormonal imbalance
History of amenorrhoea
History of eating disorders
Alcohol consumption by patient or blood relatives
Details of mother's pregnancy with patient (i.e., measures of uterine environment): Any drugs
taken, smoking, alcohol, stress levels, exposure to plastics (i.e.,Tupperware), composition of
diet (see below)
Sleep patterns: Number of hours a night, continuous/overall
Diet: Meat, organic produce, vegetables, vitamin or other supplement consumption, dairy (full
fat or reduced fat), coffee/tea consumption, folic acid, sugar (complex, artificial, simple),
processed food versus home cooked.
Exposure to plastics: Microwave in plastic, cook with plastic, store food in plastic, plastic water
or coffee mugs.
Water consumption: Amount per day, format: straight from the tap, bottled water (plastic or
glass bottle), filtered (type: e.g., Britta/Pur)
Residence history starting with mother's pregnancy: Location/duration
Environmental exposure to potential toxins for different regions (extracted from government
monitoring databases)
Health metrics: Autoimmune disease, chronic illness/condition
Pelvic surgery history
Life time number of pelvic X-rays
History of sexually transmitted infections: Type/treatment/outcome
Female reproductive hormone levels: follicle stimulating hormone (FSH), anti-Müllerian
hormone (AMH), estrogen (E2), progesterone
Stress
Thickness and type of endometrium throughout the menstrual cycle.
Age
Height
Fertility treatment history and details: History of hormone stimulation, brand of drugs used,
basal antral follicle count, follicle count after stimulation with different protocols,
number/quality/stage of retrieved oocytes/development profile of embryos resulting from in
vitro insemination (including use of ICSI), details of IVF procedure (which clinic,
doctor/embryologist at clinic, assisted hatching, fresh or thawed oocytes/embryos, embryo
transfer (blood on the catheter/squirt detection and direction on ultrasound), number of
successful and unsuccessful IVF attempts
Morning sickness during pregnancy
Breast size before/during/after pregnancy
History of ovarian cysts
Twin or sibling from multiple birth (monozygotic or dizygotic)
Semen analysis (count, motility, morphology)
Vasectomy
Testosterone levels
Date of last use and/or frequency of use of a hot tub or sauna
Blood type
Diethylstilbestrol (DES) exposure in utero
Past and current exercise/athletic history
Levels of phthalates, including metabolites:
MEP—monoethyl phthalate, MECPP—mono(2-ethyl-5-carboxypentyl) phthalate,
MEHHP—mono(2-ethyl-5-hydroxyhexyl) phthalate, MEOHP—mono(2-ethyl-5-ox-ohexyl) phthalate,
MBP—monobutyl phthalate, MBzP—monobenzyl phthalate, MEHP—mono(2-ethylhexyl)
phthalate, MiBP—mono-isobutyl phthalate, MCPP—mono(3-carboxypropyl) phthalate,
MCOP—monocarboxyisooctyl phthalate, MCNP—monocarboxyisononyl phthalate
Familial history of Premature Ovarian Failure/Primary Ovarian Insufficiency
Autoimmunity history - Antiadrenal antibodies (anti-21-hydroxylase antibodies), antiovarian
antibodies, antithyroid anitibodies (anti-thyroid peroxidase, antithyroglobulin)
Additional female hormone levels: Leutenizing hormone (using immunofluorometric assay),
Δ4-Androstenedione (using radioimmunoassay), Dehydroepiandrosterone (using
radioimmunoassay), and Inhibin B (commercial ELISA)
Number of years trying to conceive
Dioxin and PVC exposure
Hair color
Nevi (moles)
Lead, cadmium, and other heavy metal exposure
For a particular ART cycle: The percentage of eggs that were abnormally fertilized, if assisted
hatching was performed, if anesthesia was used, average number of cells contained by the
embryo at the time of cryopreservation, average degree of expansion for blastocyst represented
as a score, average degree of expansion of a previously frozen embryo represented as a score,
embryo quality metrics including but not limited to degree of cell fragmentation and
visualization of a or organization/number of cells contained in the inner cell mass (ICM), the
fraction of overall embryos that make it to the blastocyst stage of development, the number of
embryos that make it to the blastocyst stage of development, use of birth control, the brand
name of the hormones used in ovulation induction, hyperstimulation syndrome, reason for
cancelation of a treatment cycle, chemical pregnancy detected, clinical pregnancy detected,
count of germinal vesicle containing oocytes upon retrieval, count of metaphase I stage eggs
upon retrieval, count of metaphase II stage eggs upon retrieval, count of embryos or oocytes
arrested in development and the stage of development or day of development post-oocyte
retrieval, number of embryos transferred and date in days post-oocyte retrieval that the embryos
were transferred, how many embryos were cryopreserved and at what stage of development

In one embodiment, the assessment of a patient's probability of achieving an ongoing pregnancy incorporates clinical data such as age, antral follicle count, medication type, sperm motility, clinical diagnoses, BMI, hormone levels, and previous fertility treatments (including the use of ovulation induction agents).

Clinical information can be obtained by any means known in the art. In many cases this information can be obtained from a questionnaire completed by the subject that contains questions regarding certain clinical data, such as age. Additional information can be obtained from a questionnaire completed by the subject's partner and blood relatives. The questionnaire includes questions regarding the subject's clinical traits, such as her or his age, smoking habits, or frequency of alcohol consumption.

Information can also be obtained from the medical history of the subject, as well as the medical history of blood relatives and other family members, such as any clinical diagnoses, prior fertility treatments and current medications. Additional information can be obtained from the medical history and family medical history of the subject's partner. Medical history information can be obtained through analysis of electronic medical records, paper medical records, a series of questions about medical history included in the questionnaire, and a combination thereof.

In other embodiments, an assay specific to a phenotypic trait or an environmental exposure of interest is used. Such assays are known to those of skill in the art, and may be used with methods of the invention. For example, hormones, such as follicle stimulating hormone (FSH) and luteinizing hormone (LH), may be detected from a urine or blood test. Venners et al. (Hum. Reprod. 21(9): 2272-2280, 2006) reports assays for detecting estrogen and progesterone in urine and blood samples. Venners et. al. also reports assays for detecting the chemicals used in fertility treatments.

Illicit drug use may be detected from a tissue or body fluid, such as hair, urine, sweat, or blood, and there are numerous commercially available assays (LabCorp) for conducting such tests. Standard drug tests look for ten different classes of drugs, and the test is commercially known as a “10-panel urine screen.” The 10-panel urine screen consists of the following: 1. Amphetamines (including Methamphetamine) 2. Barbiturates 3. Benzodiazepines 4. Cannabinoids (THC) 5. Cocaine 6. Methadone 7. Methaqualone 8. Opiates (Codeine, Morphine, Heroin, Oxycodone, Vicodin, etc.) 9. Phencyclidine (PCP) 10. Propoxyphene. Use of alcohol can also be detected by such tests.

Numerous assays can be used to tests a patient's exposure to plastics (e.g., Bisphenol A (BPA)). BPA is most commonly found as a component of polycarbonates (about 74% of total BPA produced) and in the production of epoxy resins (about 20%). As well as being found in a myriad of products including plastic food and beverage contains (including baby and water bottles), BPA is also commonly found in various household appliances, electronics, sports safety equipment, adhesives, cash register receipts, medical devices, eyeglass lenses, water supply pipes, and many other products. Assays for testing blood, sweat, or urine for presence of BPA are described, for example, in Genuis et al. (Journal of Environmental and Public Health, Volume 2012, Article ID 185731, 10 pages, 2012).

A subject's body mass index (BMI) can be determined by first obtaining the subject's weight and height and then comparing to or inputting that information into a physical or computer-based table or chart. Body mass index (BMI) is a value derived from the mass and height of an individual that is used to quantify the amount of tissue mass (including muscle, fat, and bone) in an individual, such that the individual can be categorized as underweight, normal weight, overweight, or obese. The commonly accepted ranges can be found in Table 2 below.

TABLE 2
Commonly Accepted Body Mass Index Ranges

| Range | kg/m² |
|---|---|
| Underweight | <18.5 |
| Normal weight | 18.5-25 |
| Overweight | 25-30 |
| Obese | ≥30 |
| Obese class I | 30-34.99 |
| Obese class II | 35-39.99 |
| Obese class III | ≥40 |

Antral follicle count (AFC) can be determined through the use of ultrasound, preferably a vaginal ultrasound. Antral follicles are small follicles within the ovaries that are present during a latter stage of folliculogenesis. Antral follicle counts are often used as a proxy for ovarian reserve.

ii. Genetic Data

In one aspect of the invention, the assessment of the patient's potential for reproductive success and subsequent determination of a treatment protocol includes the use of genetic data from both the patient and a reference population. These genetic data are utilized to provide more accurate prognoses that can inform downstream diagnostic tests and treatments that may benefit the subject.

Genetic data for use with methods of the invention include any biomarkers that are associated with infertility/fertility/ability to achieve ongoing pregnancy. Exemplary biomarkers include genes (e.g., any region of DNA encoding a functional product), genetic regions (e.g., regions including genes and intergenic regions with a particular focus on regions conserved throughout evolution in placental mammals), and gene products (e.g., RNA and protein). In certain embodiments, the biomarker is an fertility-associated gene or genetic region. An fertility-associated genetic region is any DNA sequence in which variation is associated with a change in fertility. Examples of changes in fertility include, but are not limited to, the following: a homozygous mutation of an infertility-associated gene leading to a complete loss of fertility; a homozygous mutation of an infertility-associated gene that is incompletely penetrant leading to reduction in fertility that varies from individual to individual; a recessive mutation in heterozygous, having no effect on fertility; a dominant mutation in heterozygous, leading to a fertility phenotype; and the infertility-associated gene is X-linked, such that a potential defect in fertility depends on whether a non-functional allele of the gene is located on an inactive X chromosome (Barr body) or on an expressed X chromosome.

In particular embodiments, the assessed fertility-associated genetic region is a maternal effect gene. Maternal effect genes are genes that have been found to encode key structures and functions in mammalian oocytes (Yurttas et al., Reproduction 139:809-823, 2010). Maternal effect genes are described, for example in, Christians et al. (Mol Cell Biol 17:778-88, 1997); Christians et al., Nature 407:693-694, 2000); Xiao et al. (EMBO J 18:5943-5952, 1999); Tong et al. (Endocrinology 145:1427-1434, 2004); Tong et al. (Nat Genet 26:267-268, 2000); Tong et al. (Endocrinology, 140:3720-3726, 1999); Tong et al. (Hum Reprod 17:903-911, 2002); Ohsugi et al. (Development 135:259-269, 2008); Borowczyk et al. (Proc Natl Acad Sci USA., 2009); and Wu (Hum Reprod 24:415-424, 2009). Maternal effect genes are also described in U.S. Ser. No. 12/889,304. The content of each of these is incorporated by reference herein in its entirety.

In particular embodiments, the fertility-associated genetic region is one or more genes (including exons, introns, and 10 kb of DNA flanking either side of said gene) selected from the genes shown in Table 3 below. In Table 3, OMIM reference numbers are provided when available.

TABLE 3
Human Infertility-Related Genes (OMIM #)

| | | | |
|---|---|---|---|
| ABCA1 (600046) | ACTL6A (604958) | ACTL8 | ACVR1 (102576) |
| ACVR1B (601300) | ACVR1C (608981) | ACVR2(102581) | ACVR2A (102581) |
| ACVR2B (602730) | ACVRL1 (601284) | ADA (608958) | ADAMTS1 (605174) |
| ADM (103275) | ADM2 (608682) | AFF2 (300806) | AGT (106150) |
| AHR (600253) | AIRE (607358) | AK2 (103020) | AK7 |
| AKR1C1 (600449) | AKR1C2 (600450) | AKR1C3 (603966) | AKR1C4 (600451) |
| AKT1 (164730) | ALDOA (103850) | ALDOB (612724) | ALDOC (103870) |
| ALPL (171760) | AMBP (176870) | AMD1 (180980) | AMH (600957) |
| AMHR2 (600956) | ANK3 (600465) | ANXA1 (151690) | APC (611731) |
| APOA1 (107680) | APOE (107741) | AQP4 (600308) | AR (313700) |
| AREG (104640) | ARF1 (103180) | ARF3 (103190) | ARF4 (601177) |
| ARF5 (103188) | ARFRP1 (604699) | ARL1 (603425) | ARL10 (612405) |
| ARL11 (609351) | ARL13A | ARL13B (608922) | ARL15 |
| ARL2 (601175) | ARL3 (604695) | ARL4A (604786) | ARL4C (604787) |
| ARL4D (600732) | ARL5A (608960) | ARL5B (608909) | ARL5C |
| ARL6 (608845) | ARL8A | ARL8B | ARMC2 |
| ARNTL (602550) | ASCL2 (601886) | ATF7IP (613644) | ATG7 (608760) |
| ATM (607585) | ATR (601215) | ATXN2 (601517) | AURKA (603072) |
| AURKB (604970) | AUTS2 (607270) | BARD1 (601593) | BAX (600040) |
| BBS1 (209901) | BBS10 (610148) | BBS12 (610683) | BBS2 (606151) |
| BBS4 (600374) | BBS5 (603650) | BBS7 (607590) | BBS9 (607968) |
| BCL2 (151430) | BCL2L1 (600039) | BCL2L10 (606910) | BDNF (113505) |
| BECN1 (604378) | BHMT (602888) | BLVRB (600941) | BMP15 (300247) |
| BMP2 (112261) | BMP3 (112263) | BMP4 (112262) | BMP5 (112265) |
| BMP6 (112266) | BMP7 (112267) | BMPR1A (601299) | BMPR1B (603248) |
| BMPR2 (600799) | BNC1 (601930) | BOP1 (610596) | BRCA1 (113705) |
| BRCA2 (600185) | BRIP1 (605882) | BRSK1 (609235) | BRWD1 |
| BSG (109480) | BTG4 (605673) | BUB1 (602452) | BUB1B (602860) |
| C2orf86 (613580) | C3 (120700) | C3orf56 | C6orf221 (611687) |
| CA1 (114800) | CARD8 (609051) | CARM1 (603934) | CASP1 (147678) |
| CASP2 (600639) | CASP5 (602665) | CASP6 (601532) | CASP8 (601763) |
| CBS (613381) | CBX1 (604511) | CBX2 (602770) | CBX5 (604478) |
| CCDC101 (613374) | CCDC28B (610162) | CCL13 (601391) | CCL14 (601392) |
| CCL4 (182284) | CCF5 (187011) | CCL8 (602283) | CCND1 (168461) |
| CCND2 (123833) | CCND3 (123834) | CCNH (601953) | CCS (603864) |
| CD19 (107265) | CD24 (600074) | CD55 (125240) | CD81 (186845) |
| CD9 (143030) | CDC42 (116952) | CDK4 (123829) | CDK6 (603368) |
| CDK7 (601955) | CDKN1B (600778) | CDKN1C (600856) | CDKN2A (600160) |
| CDX2 (600297) | CDX4 (300025) | CEACAM20 | CEBPA (116897) |
| CEBPB (189965) | CEBPD (116898) | CEBPE (600749) | CEBPG (138972) |
| CEBPZ (612828) | CELF1 (601074) | CELF4 (612679) | CENPB (117140) |
| CENPF (600236) | CENPI (300065) | CEP290 (610142) | CFC1 (605194) |
| CGA (118850) | CGB (118860) | CGB1 (608823) | CGB2 (608824) |
| CGB5 (608825) | CHD7 (608892) | CHST2 (603798) | CLDN3 (602910) |
| COIL (600272) | COL1A2 (120160) | COL4A3BP (604677) | COMT (116790) |
| COPE (606942) | COX2 (600262) | CP (117700) | CPEB1 (607342) |
| CRHR1 (122561) | CRYBB2 (123620) | CSF1 (120420) | CSF2 (138960) |
| CSTF1 (600369) | CSTF2 (600368) | CTCF (604167) | CTCFL (607022) |
| CTF2P | CTGF (121009) | CTH (607657) | CTNNB1 (116806) |
| CUL1 (603134) | CX3CL1 (601880) | CXCL10 (147310) | CXCL9 (601704) |
| CXorf67 | CYP11A1 (118485) | CYP11B1 (610613) | CYP11B2 (124080) |
| CYP17A1 (609300) | CYP19A1 (107910) | CYP1A1 (108330) | CYP27B1 (609506) |
| DAZ2 (400026) | DAZL (601486) | DCTPP1 | DDIT3 (126337) |
| DDX11 (601150) | DDX20 (606168) | DDX3X (300160) | DDX43 (606286) |
| DEPDC7 (612294) | DHFR (126060) | DHFRL1 | DIAPH2 (300108) |
| DICER1 (606241) | DKK1 (605189) | DLC1 (604258) | DLGAP5 |
| DMAP1 (605077) | DMC1 (602721) | DNAJB1 (604572) | DNMT1 (126375) |
| DNMT3B (602900) | DPPA3 (608408) | DPPA5 (611111) | DPYD (612779) |
| DTNBP1 (607145) | DYNLL1 (601562) | ECHS1 (602292) | EEF1A1 (130590) |
| EEF1A2 (602959) | EFNA1 (191164) | EFNA2 (602756) | EFNA3 (601381) |
| EFNA4 (601380) | EFNA5 (601535) | EFNB1 (300035) | EFNB2 (600527) |
| EFNB3 (602297) | EGR1 (128990) | EGR2 (129010) | EGR3 (602419) |
| EGR4 (128992) | EHMT1 (607001) | EHMT2 (604599) | EIF2B2 (606454) |
| EIF2B4 (606687) | EIF2B5 (603945) | EIF2C2 (606229) | EIF3C (603916) |
| EIF3CL (603916) | EPHA1 (179610) | EPHA10 (611123) | EPHA2 (176946) |
| EPHA3 (179611) | EPHA4 (602188) | EPHA5 (600004) | EPHA6 (600066) |
| EPHA7 (602190) | EPHA8 (176945) | EPHB1 (600600) | EPHB2 (600997) |
| EPHB3 (601839) | EPHB4 (600011) | EPHB6 (602757) | ERCC1 (126380) |
| ERCC2 (126340) | EREG (602061) | ESR1 (133430) | ESR2 (601663) |
| ESR2 (601663) | ESRRB (602167) | ETV5 (601600) | EZH2 (601573) |
| EZR (123900) | FANCC (613899) | FANCG (602956) | FANCL (608111) |
| FAR1 | FAR2 | FASLG (134638) | FBN1 (134797) |
| FBN2 (612570) | FBN3 (608529) | FBRS (608601) | FBRSF1 |
| FBXO10 (609092) | FBXO11 (607871) | FCRL3 (606510) | FDXR (103270) |
| FGF23 (605380) | FGF8 (600483) | FGFBP1 (607737) | FGFBP3 |
| FGFR1 (136350) | FHL2 (602633) | FIGLA (608697) | FILIP1L (612993) |
| FKBP4 (600611) | FMN2 (606373) | FMR1 (309550) | FOLR1 (136430) |
| FOLR2 (136425) | FOXE1 (602617) | FOXF2 (605597) | FOXN1 (600838) |
| FOXO3 (602681) | FOXP3 (300292) | FRZB (605083) | FSHB (136530) |
| FSHR (136435) | FST (136470) | GALT (606999) | GBP5 (611467) |
| GCK (138079) | GDF1 (602880) | GDF3 (606522) | GDF9 (601918) |
| GGT1 (612346) | GJA1 (121014) | GJA10 (611924) | GJA3 (121015) |
| GJA4 (121012) | GJA5 (121013) | GJA8 (600897) | GJB1 (304040) |
| GJB2 (121011) | GJB3 (603324) | GJB4 (605425) | GJB6 (604418) |
| GJB7 (611921) | GJC1 (608655) | GJC2 (608803) | GJC3 (611925) |
| GJD2 (607058) | GJD3 (607425) | GJD4 (611922) | GNA13 (604406) |
| GNB2 (139390) | GNRH1 (152760) | GNRH2 (602352) | GNRHR (138850) |
| GPC3 (300037) | GPRC5A (604138) | GPRC5B (605948) | GREM2 (608832) |
| GRN (138945) | GSPT1 (139259) | GSTA1 (138359) | H19 (103280) |
| H1FOO (142709) | HABP2 (603924) | HADHA (600890) | HAND2 (602407) |
| HBA1 (141800) | HBA2 (141850) | HBB (141900) | HELLS (603946) |
| HK3 (142570) | HMOX1 (141250) | HNRNPK (600712) | HOXA11 (142958) |
| HPGD (601688) | HS6ST1 (604846) | HSD17B1 (109684) | HSD17B12 (609574) |
| HSD17B2 (109685) | HSD17B4 (601860) | HSD17B7 (606756) | HSD3B1 (109715) |
| HSF1 (140580) | HSF2BP (604554) | HSP90B1 (191175) | HSPG2 (142461) |
| HTATIP2 (605628) | ICAM1 (147840) | ICAM2 (146630) | ICAM3 (146631) |
| IDH1 (147700) | IFI30 (604664) | IFITM1 (604456) | IGF1 (147440) |
| IGF1R (147370) | IGF2 (147470) | IGF2BP1 (608288) | IGF2BP2 (608289) |
| IGF2BP3 (608259) | IGF2BP3 (608259) | IGF2R (147280) | IGFALS (601489) |
| IGFBP1 (146730) | IGFBP2 (146731) | IGFBP3 (146732) | IGFBP4 (146733) |
| IGFBP5 (146734) | IGFBP6 (146735) | IGFBP7 (602867) | IGFBPL1 (610413) |
| IL10 (124092) | IL11RA (600939) | IL12A (161560) | IL12B (161561) |
| IL13 (147683) | IL17A (603149) | IL17B (604627) | IL17C (604628) |
| IL17D (607587) | IL17F (606496) | IL1A (147760) | IL1B (147720) |
| IL23A (605580) | IL23R (607562) | IL4 (147780) | IL5 (147850) |
| IL5RA (147851) | IL6 (147620) | IL6ST (600694) | IL8 (146930) |
| ILK (602366) | INHA (147380) | INHBA (147290) | INHBB (147390) |
| IRF1 (147575) | ISG15 (147571) | ITGA11 (604789) | ITGA2 (192974) |
| ITGA3 (605025) | ITGA4 (192975) | ITGA7 (600536) | ITGA9 (603963) |
| ITGAV (193210) | ITGB1 (135630) | JAG1 (601920) | JAG2 (602570) |
| JARID2 (601594) | JMY (604279) | KAL1 (300836) | KDM1A (609132) |
| KDM1B (613081) | KDM3A (611512) | KDM4A (609764) | KDM5A (180202) |
| KDM5B (605393) | KHDC1 (611688) | KIAA0430 (614593) | KIF2C (604538) |
| KISS1 (603286) | KISS1R (604161) | KITLG (184745) | KL (604824) |
| KLF4 (602253) | KLF9 (602902) | KLHL7 (611119) | LAMC1 (150290) |
| LAMC2 (150292) | LAMP1 (153330) | LAMP2 (309060) | LAMP3 (605883) |
| LDB3 (605906) | LEP (164160) | LEPR (601007) | LFNG (602576) |
| LHB (152780) | LHCGR (152790) | LHX8 (604425) | LIF (159540) |
| LIFR (151443) | LIMS1 (602567) | LIMS2 (607908) | LIMS3 |
| LIMS3L | LIN28 (611043) | LIN28B (611044) | LMNA (150330) |
| LOC613037 | LOXL4 (607318) | LPP (600700) | LYRM1 (614709) |
| MAD1L1 (602686) | MAD2L1 (601467) | MAD2L1BP | MAF (177075) |
| MAP3K1 (600982) | MAP3K2 (609487) | MAPK1 (176948) | MAPK3 (601795) |
| MAPK8 (601158) | MAPK9 (602896) | MB21D1 (613973) | MBD1 (156535) |
| MBD2 (603547) | MBD3 (603573) | MBD4 (603574) | MCL1 (159552) |
| MCM8 (608187) | MDK (162096) | MDM2 (164785) | MDM4 (602704) |
| MECP2 (300005) | MED12 (300188) | MERTK (604705) | METTL3 (612472) |
| MGAT1 (160995) | MITF (156845) | MKKS (604896) | MKS1 (609883) |
| MLH1 (120436) | MLH3 (604395) | MOS (190060) | MPPED2 (600911) |
| MRS2 | MSH2 (609309) | MSH3 (600887) | MSH4 (602105) |
| MSH5 (603382) | MSH6 (600678) | MST1 (142408) | MSX1 (142983) |
| MSX2 (123101) | MTA2 (603947) | MTHFD1 (172460) | MTHFR (607093) |
| MTO1 (614667) | MTOR (601231) | MTRR (602568) | MUC4 (158372) |
| MVP (605088) | MX1 (147150) | MYC (190080) | NAB1 (600800) |
| NAB2 (602381) | NAT1 (108345) | NCAM1 (116930) | NCOA2 (601993) |
| NCOR1 (600849) | NCOR2 (600848) | NDP (300658) | NFE2L3 (604135) |
| NLRP1 (606636) | NLRP10 (609662) | NLRP11 (609664) | NLRP12 (609648) |
| NLRP13 (609660) | NLRP14 (609665) | NLRP2 (609364) | NLRP3 (606416) |
| NLRP4 (609645) | NLRP5 (609658) | NLRP6 (609650) | NLRP7 (609661) |
| NLRP8 (609659) | NLRP9 (609663) | NNMT (600008) | NOBOX (610934) |
| NODAL (601265) | NOG (602991) | NOS3 (163729) | NOTCH1 (190198) |
| NOTCH2 (600275) | NPM2 (608073) | NPR2 (108961) | NR2C2 (601426) |
| NR3C1 (138040) | NR5A1 (184757) | NR5A2 (604453) | NRIP1 (602490) |
| NRIP2 | NRIP3 (613125) | NTF4 (162662) | NTRK1 (191315) |
| NTRK2 (600456) | NUPR1 (614812) | OAS1 (164350) | OAT (613349) |
| OFD1 (300170) | OOEP (611689) | ORAI1 (610277) | OTC (300461) |
| PADI1 (607934) | PADI2 (607935) | PADI3 (606755) | PADI4 (605347) |
| PADI6 (610363) | PAEP (173310) | PAIP1 (605184) | PARP12 (612481) |
| PCNA (176740) | PCP4L1 | PDE3A (123805) | PDK1 (602524) |
| PGK1 (311800) | PGR (607311) | PGRMC1 (300435) | PGRMC2 (607735) |
| PIGA (311770) | PIM1 (164960) | PLA2G2A (172411) | PLA2G4C (603602) |
| PLA2G7 (601690) | PLAC1L | PLAG1 (603026) | PLAGL1 (603044) |
| PLCB1 (607120) | PMS1 (600258) | PMS2 (600259) | POF1B (300603) |
| POLG (174763) | POLR3A (614258) | POMZP3 (600587) | POU5F1 (164177) |
| PPID (601753) | PPP2CB (176916) | PRDM1 (603423) | PRDM9 (609760) |
| PRKCA (176960) | PRKCB (176970) | PRKCD (176977) | PRKCDBP |
| PRKCE (176975) | PRKCG (176980) | PRKCQ (600448) | PRKRA (603424) |
| PRLR (176761) | PRMT1 (602950) | PRMT10 (307150) | PRMT2 (601961) |
| PRMT3 (603190) | PRMT5 (604045) | PRMT6 (608274) | PRMT7 (610087) |
| PRMT8 (610086) | PROK1 (606233) | PROK2 (607002) | PROKR1 (607122) |
| PROKR2 (607123) | PSEN1 (104311) | PSEN2 (600759) | PTGDR (604687) |
| PTGER1 (176802) | PTGER2 (176804) | PTGER3 (176806) | PTGER4 (601586) |
| PTGES (605172) | PTGES2 (608152) | PTGES3 (607061) | PTGFR (600563) |
| PTGFRN (601204) | PTGS1 (176805) | PTGS2 (600262) | PTN (162095) |
| PTX3 (602492) | QDPR (612676) | RAD17 (603139) | RAX (601881) |
| RBP4 (180250) | RCOR1 (607675) | RCOR2 | RCOR3 |
| RDH11 (607849) | REC8 (608193) | REXO1 (609614) | REXO2 (607149) |
| RFPL4A (612601) | RGS2 (600861) | RGS3 (602189) | RSPO1 (609595) |
| RTEL1 (608833) | SAFB (602895) | SAR1A (607691) | SAR1B (607690) |
| SCARB1 (601040) | SDC3 (186357) | SELL (153240) | SEPHS1 (600902) |
| SEPHS2 (606218) | SERPINA10 (605271) | SFRP1 (604156) | SFRP2 (604157) |
| SFRP4 (606570) | SFRP5 (604158) | SGK1 (602958) | SGOL2 (612425) |
| SH2B1 (608937) | SH2B2 (605300) | SH2B3 (605093) | SIRT1 (604479) |
| SIRT2 (604480) | SIRT3 (604481) | SIRT4 (604482) | SIRT5 (604483) |
| SIRT6 (606211) | SIRT7 (606212) | SLC19A1 (600424) | SLC28A1 (606207) |
| SLC28A2 (606208) | SLC28A3 (608269) | SLC2A8 (605245) | SLC6A2 (163970) |
| SLC6A4 (182138) | SLCO2A1 (601460) | SLITRK4 (300562) | SMAD1 (601595) |
| SMAD2 (601366) | SMAD3 (603109) | SMAD4 (600993) | SMAD5 (603110) |
| SMAD6 (602931) | SMAD7 (602932) | SMAD9 (603295) | SMARCA4 (603254) |
| SMARCA5 (603375) | SMC1A (300040) | SMC1B (608685) | SMC3 (606062) |
| SMC4 (605575) | SMPD1 (607608) | SOCS1 (603597) | SOD1 (147450) |
| SOD2 (147460) | SOD3 (185490) | SOX17 (610928) | SOX3 (313430) |
| SPAG17 | SPARC (182120) | SPIN1 (609936) | SPN (182160) |
| SPO11 (605114) | SPP1 (166490) | SPSB2 (611658) | SPTB (182870) |
| SPTBN1 (182790) | SPTBN4 (606214) | SRCAP (611421) | SRD5A1 (184753) |
| SRSF4 (601940) | SRSF7 (600572) | ST5 (140750) | STAG3 (608489) |
| STAR (600617) | STARD10 | STARD13 (609866) | STARD3 (607048) |
| STARD3NL (611759) | STARD4 (607049) | STARD5 (607050) | STARD6 (607051) |
| STARD7 | STARD8 (300689) | STARD9 (614642) | STAT1 (600555) |
| STAT2 (600556) | STAT3 (102582) | STAT4 (600558) | STAT5A (601511) |
| STAT5B (604260) | STAT6 (601512) | STC1 (601185) | STIM1 (605921) |
| STK3 (605030) | SULT1E1 (600043) | SUZ12 (606245) | SYCE1 (611486) |
| SYCE2 (611487) | SYCP1 (602162) | SYCP2 (604105) | SYCP3 (604759) |
| SYNE1 (608441) | SYNE2 (608442) | TAC3 (162330) | TACC3 (605303) |
| TACR3 (162332) | TAF10 (600475) | TAF3 (606576) | TAF4 (601796) |
| TAF4B (601689) | TAF5 (601787) | TAF5L | TAF8 (609514) |
| TAF9 (600822) | TAP1 (170260) | TBL1X (300196) | TBXA2R (188070) |
| TCL1A (186960) | TCL1B (603769) | TCL6 (604412) | TCN2 (613441) |
| TDGF1 (187395) | TERC (602322) | TERF1 (600951) | TERT (187270) |
| TEX12 (605791) | TEX9 | TF (190000) | TFAP2C (601602) |
| TFPI (152310) | TFPI2 (600033) | TG (188450) | TGFB1 (190180) |
| TGFB1I1 (602353) | TGFBR3 (600742) | THOC5 (612733) | THSD7B |
| TLE6 (612399) | TM4SF1 (191155) | TMEM67 (609884) | TNF (191160) |
| TNFAIP6 (600410) | TNFSF13B (603969) | TOP2A (126430) | TOP2B (126431) |
| TP53 (191170) | TP53I3 (605171) | TP63 (603273) | TP73 (601990) |
| TPMT (187680) | TPRXL (611167) | TPT1 (600763) | TRIM32 (602290) |
| TSC2 (191092) | TSHB (188540) | TSIX (300181) | TTC8 (608132) |
| TUBB4Q (158900) | TUFM (602389) | TYMS (188350) | UBB (191339) |
| UBC (191340) | UBD (606050) | UBE2D3 (602963) | UBE3A (601623) |
| UBL4A (312070) | UBL4B (611127) | UIMC1 (609433) | UQCR11 (609711) |
| UQCRC2 (191329) | USP9X (300072) | VDR (601769) | VEGFA (192240) |
| VEGFB (601398) | VEGFC (601528) | VHL (608537) | VIM (193060) |
| VKORC1 (608547) | VKORC1L1 (608838) | WAS (300392) | WISP2 (603399) |
| WNT7A (601570) | WNT7B (601967) | WT1 (607102) | XDH (607633) |
| XIST (314670) | YBX1 (154030) | YBX2 (611447) | ZAR1 (607520) |
| ZFX (314980) | ZNF22 (194529) | ZNF267 (604752) | ZNF689 |
| ZNF720 | ZNF787 | ZNF84 | ZP1 (195000) |
| ZP2 (182888) | ZP3 (182889) | ZP4 (613514) | |

The genes listed in Table 3 can be involved in different aspects of reproduction/fertility related processes. Furthermore, additional genes beyond those maternal effect genes listed in Table 3 can also affect fertility.

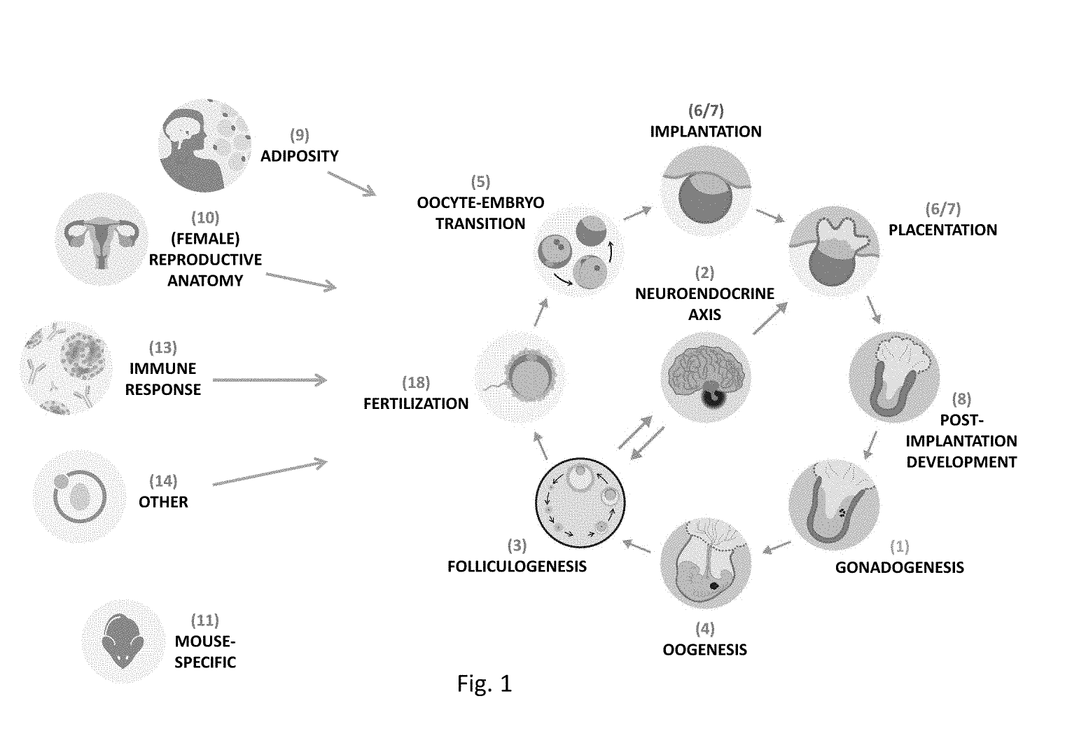

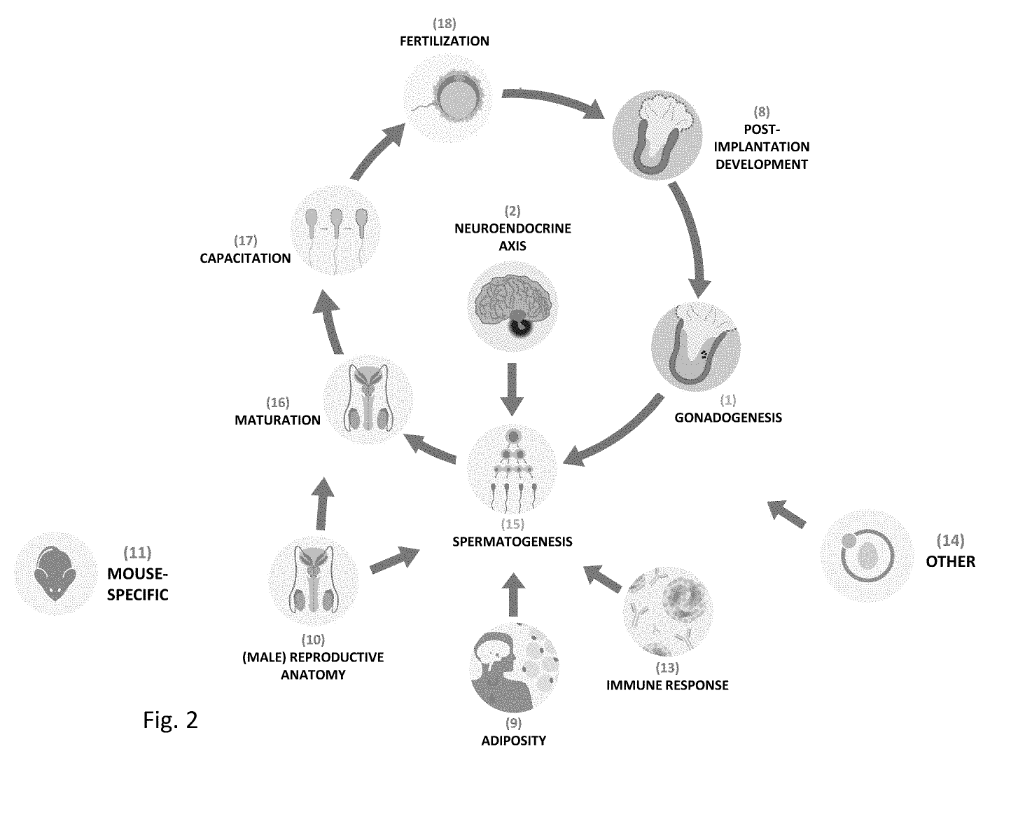

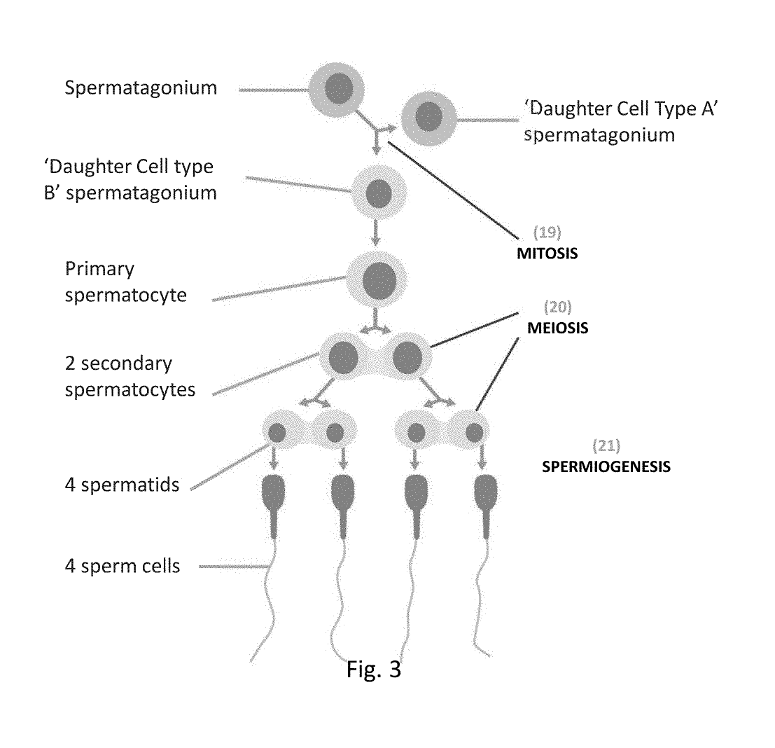

Genes affecting fertility can be involved with a number of male- and female-specific processes, or functional biological classifications, such as those shown in FIGS. 1-3. As shown in FIG. 1, female reproductive/fertility-related processes, or classifications, include gonadogenesis, neuroendocrine axis, folliculogensis, oogenesis, oocyte-embyro transition, placentation, post-implantation development, adiposity, (female) reproductive anatomy, immune response, fertilization and other processes. Male reproductive/fertility-related processes, or classifications, include gonadogenesis neuroendocrine axis, post-implantation development, adiposity, (male) reproductive anatomy, immune response, spermatogenesis, sperm maturation and capacitation, fertilization, mitosis, meiosis, spermiogenesis, and other processes, as shown in FIGS. 2 and 3. These processes are described in more detail below.

Gonadogenesis encompasses the processes regulating the development of the ovaries and testes, and involves, but is not limited to, primordial germ cell specification and proliferation. The neuroendocrine axis encompasses for example the physiological pathways and structures regulating the production and activity of hormones in a number of different tissues in the human body, including the brain and gonads. Folliculogenesis encompasses the physiological mechanisms regulating the development of primordial follicles to cystic follicles in the ovary. Oogenesis encompasses the physiological mechanisms regulating the development of primordial oocytes to mature meiosis-II stage oocytes ready to be fertilized, hence those that are specific to female reproductive biology. Oocyte-embryo transition encompasses the physiological mechanisms regulating the development of the early embryo and includes mechanisms related to egg quality, such as oocyte cytoplasmic lattice formation, and paternal effect mechanisms. Placentation (Embryonic) encompasses the embryo-specific physiological mechanisms regulating implantation and the development of the placenta. Placentation (Uterine) encompasses the uterus-specific physiological mechanisms regulating embryo implantation and the development of the placenta. Post-implantation development encompasses the physiological mechanisms regulating post-implantation embryo development, particularly those whose disruption might lead to abnormal development or pregnancy loss in humans. Adiposity encompasses the physiological mechanisms regulating adipose tissue and body weight, which are known to play an important, indirect role in mammalian fecundity and infertility. Reproductive anatomy encompasses any phenotype relating to anatomical changes that could impact reproduction, fecundity, or fertility. Immune response encompasses phenotypes that are specific to aspects of immune response mechanisms, which are known to play an important role in mammalian reproduction and fertility.

Spermatogenesis encompasses the processes involved in the production or development of mature spermatozoa, hence those that are specific to male reproductive biology. Maturation encompasses processes that enable spermatozoa to fertilize eggs, hence those that are specific to male reproductive biology. Capacitation encompasses processes specific to functional capacitation of spermatozoa in the vaginal canal and uterus. Fertilization encompasses processes relating to the union of a human egg and sperm. Mitosis encompasses the cell division processes that end with two daughter cells that have the same chromosomal complement as the parent cell. Alterations to the mitotic processes may affect fertility-related cell proliferation or tissue maintenance. Meiosis encompasses processes regulating cell division such that it results in four daughter cells each with exactly half the chromosome complement of the parent cell, for example during gametogenesis. Spermiogenesis encompasses processes regulating the morphological differentiation of haploid cells into sperm.

Mutations in genes associated with these various processes result in fertility difficulties for individuals containing these mutations and can affect an individual's potential for reproductive success.

iii. Obtaining Genetic Data

Genetic data can be obtained, for example, by conducting an assay on a sample from a male or female that detects either a mutation in an infertility-associated genetic region or abnormal (over or under) expression of an infertility-associated genetic region of the individual. The presence of certain mutations in those genetic regions or abnormal expression levels of those genetic regions is indicative fertility outcomes, i.e., the potential for reproductive success. Exemplary mutations include, but are not limited to, a single nucleotide polymorphism, a deletion, an insertion, an inversion, a genetic rearrangement, a copy number variation, or a combination thereof.

A sample may include a human tissue or bodily fluid and may be collected in any clinically acceptable manner. A tissue is a mass of connected cells and/or extracellular matrix material, e.g., skin tissue, hair, nails, nasal passage tissue, central nervous system tissue, neural tissue, eye tissue, liver tissue, kidney tissue, placental tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CSF. A sample may also be a fine needle aspirate or biopsied tissue, e.g, an endometrial aspirate, breast tissue biopsy, and the like. A sample also may be media containing cells or biological material. A sample may also be a blood clot, for example, a blood clot that has been obtained from whole blood after the serum has been removed. In certain embodiments, the sample may include reproductive cells or tissues, such as gametic cells, gonadal tissue, fertilized embryos, and placenta. In certain embodiments, the sample is blood, saliva, or semen collected from the subject. In some aspects, the sample is the same sample obtained for analysis of the individual's microbiome.

Genetic information from the sample can be obtained by nucleic acid extraction from the sample, as described above with respect to analysis of microorganisms. In particular embodiments, the assay is conducted on fertility-related genes or genetic regions containing the gene or a part thereof, such as those genes found in Table 3. Detailed descriptions of conventional methods, such as those employed to make and use nucleic acid arrays, amplification primers, hybridization probes, and the like can be found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.), Applied Biosystems (Foster City, Calif.), and Agilent Technologies (Santa Clara, Calif.).

Methods of detecting variations (e.g., mutations) are known in the art. In certain embodiments, a known single nucleotide polymorphism at a particular position can be detected by single base extension for a primer that binds to the sample DNA adjacent to that position. See for example Shuber et al. (U.S. Pat. No. 6,566,101), the content of which is incorporated by reference herein in its entirety. In other embodiments, a hybridization probe might be employed that overlaps the SNP of interest and selectively hybridizes to sample nucleic acids containing a particular nucleotide at that position. See for example Shuber et al. (U.S. Pat. Nos. 6,214,558 and 6,300,077), the content of which is incorporated by reference herein in its entirety.

In particular embodiments, nucleic acids are sequenced in order to detect variants in the nucleic acid compared to wild-type and/or non-mutated forms of the sequence. The nucleic acid can include a plurality of nucleic acids derived from a plurality of genetic elements. Methods of detecting sequence variants are known in the art, and sequence variants can be detected by any sequencing method known in the art, such as those described above with respect to the sequencing of nucleic acid from microorganisms.

As noted with respect to the identification of microorganisms, sequencing by any of the methods described above and known in the art produces sequence reads. Sequence reads can be analyzed to call variants by any number of methods known in the art. Sequence reads are aligned to a microbial reference genome set (e.g., HOMD reference genome of annotated oral microbiome species) using Burrows-Wheeler Aligner (BWA), an alignment algorithm. See, background Li & Durbin, 2009, Fast and accurate short read alignment with Burrows-Wheeler Transform. Bioinformatics 25:1754-60 and McKenna et al., 2010. Thereafter, single base changes in aligned reads relative to the reference genome (or vice versa) are reported as single nucleotide polymorphisms (SNPs). An example of a tool used for calling variants is the Genome Analysis Toolkit (GATK), a software package developed for calling variants in high throughput sequencing data. See The Genome Analysis Toolkit: a MapReduce framework for analyzing next-generation DNA sequencing data, Genome Res 20(9):1297-1303, the contents of each of which are incorporated by reference.

GATK variant calling results are reported in a format known as Variant Call Format (VCF). The VCF format is described in Danecek et al., 2011, The variant call format and VCFtools, Bioinformatics 27(15): 2156-2158. Further discussion may be found in U.S. Pub. 2013/0073214; U.S. Pub. 2013/0345066; U.S. Pub. 2013/0311106; U.S. Pub. 2013/0059740; U.S. Pub. 2012/0157322; U.S. Pub. 2015/0057946 and U.S. Pub. 2015/0056613, each incorporated by reference.

Furthermore, in certain embodiments, methods of the invention include conducting an assay on a sample from a subject that detects an abnormal (over or under) expression of an infertility-associated gene (e.g., a differentially or abnormally expressed gene). A differentially or abnormally expressed gene refers to a gene whose expression is activated to a higher or lower level in a subject suffering from a disorder, such as infertility, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as infertility, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

Methods used to detect differential gene expression in high throughput sequencing data across samples sets include DESeq2, Anders S and Huber W (2010). “Differential expression analysis for sequence count data.” Genome Biology, 11, pp. R106. doi: 10.1186/gb-2010-11-10-r106, and edgeR, Robinson M D, McCarthy D J and Smyth G K (2010). “edgeR: a Bioconductor package for differential expression analysis of digital gene expression data.” Bioinformatics, 26, pp.-1.

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999); RNAse protection assays (Hod, Biotechniques 13:852 854 (1992); and PCR-based methods, such as reverse transcription polymerase chain reaction (RT-PCR) (Weis et al., Trends in Genetics 8:263 264 (1992); the contents of all of which are incorporated by reference herein in their entirety. Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

In certain embodiments, reverse transcription PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure. Various methods are well known in the art. See, e.g., Ausubel et al., Current Protocols of Molecular Biology, John Wiley and Sons (1997); Rupp and Locker, Lab Invest. 56:A67 (1987), and De Andres et al., BioTechniques 18:42044 (1995); Held et al., Genome Research 6:986 994 (1996), the contents of which are incorporated by reference herein in their entirety.

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In another embodiment, a MassARRAY-based gene expression profiling method is used to measure gene expression. For further details see, e.g., Ding and Cantor, Proc. Natl. Acad. Sci. USA 100:3059 3064 (2003), incorporated herein by reference.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269); see also Schena et al., Proc. Natl. Acad. Sci. USA 93(2):106 149 (1996), the content of each of which is incorporated by reference herein in their entirety. Microarray analysis can be performed by commercially available equipment, following manufacturer's protocols, such as by using the Affymetrix GenChip technology, or Incyte's microarray technology.

In another aspect, protein levels can be determined by constructing an antibody microarray in which binding sites comprise immobilized, preferably monoclonal, antibodies specific to a plurality of protein species encoded by the cell genome. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes).

In yet another aspect, levels of transcripts of marker genes in a number of tissue specimens may be characterized using a “tissue array” (Kononen et al., Nat. Med 4(7):844-7 (1998)). In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. For more details see, e.g., Velculescu et al., Science 270:484 487 (1995); and Velculescu et al., Cell 88:243 51 (1997, the contents of each of which are incorporated by reference herein in their entirety).

In other embodiments, Massively Parallel Signature Sequencing (MPSS) is used to measure gene expression. For more details see, e.g., Brenner et al., Nature Biotechnology 18:630 634 (2000).

Immunohistochemistry methods are also suitable for detecting the expression levels of the gene products of the present invention. In these methods, antibodies (monoclonal or polyclonal) or antisera, such as polyclonal antisera, specific for each marker are used to detect expression. Immunohistochemistry protocols and kits are well known in the art and are commercially available.

In certain embodiments, a proteomics approach is used to measure gene expression. Proteomics typically includes the following steps: (1) separation of individual proteins in a sample by 2-D gel electrophoresis (2-D PAGE); (2) identification of the individual proteins recovered from the gel, e.g., by mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See, for example, U.S. Pat. Nos. 6,925,389; 6,989,100; and 6,890,763, each of which is incorporated by reference herein in their entirety.

iv. Incorporation of Clinical and/or Genetic Data into Analysis

In certain aspects, in addition to the analysis of the individual's microbiome, or aspects thereof, methods for assessing an individual's potential for reproductive success further involve the use of clinical and/or genetic data. Specifically, the methods can include the determination of one or more correlations between clinical and/or genetic characteristics of the individual and known pregnancy and infertility-related outcomes from a reference set of data to provide for and/or adjust the model representative of the potential for reproductive success.

Clinical characteristics obtained from the reference population include, but are not limited to, any or all of the characteristics described above in the “Clinical Data” section. Exemplary characteristics include BMI, fertility treatment history, age, antral follicle count, sperm motility, clinical diagnoses, and medication type. With respect to fertility treatment history, the reference set of data includes information as to what fertility treatments were used. Exemplary fertility treatments include, but are not limited to, assisted reproductive technologies (ART), non-ART fertility treatments (RE), and fertility preservation technologies (egg, embryo, or ovarian preservation). Exemplary assisted reproductive technologies include, without limitation, in vitro fertilization (IVF), zygote intrafallopian transfer (ZIFT), gametic intrafallopian transfer (GIFT), or intracytoplasmic sperm injection (ICSI) paired with one of the methods above. Exemplary non-ART fertility treatments include ovulation induction protocols with or without intrauterine insemination (IUI) with sperm. Exemplary ovulation induction agents include gonadotropins such as luteinizing hormone (LH), follicle stimulating hormone (FSH), and human chorionic gonadotropin (hCG); and oral ovulation induction agents such as letrozole, clomiphene citrate, bromocriptine, metformin, and cabergoline.

As with the microbiome data, the clinical characteristics obtained from the reference population is passed through the association analysis in order to determine whether and to what extent the characteristics obtained from the subjects in the reference population are associated with the potential for reproductive success.

In one embodiment, the methods also incorporate genetic characteristics from the reference population and their impact on the individual's potential for reproductive success. In certain aspects, variants within genes and genetic regions, such as those described above, are first identified. In a preferred embodiment, whole genome sequencing is conducted on DNA extracted from whole blood samples using the Illumina HiSeq platform. As described above, variants can be called using standard Genome Analysis Toolkit (GATK) methods.

Once the variants are called, a customized pipeline is used to identify deleterious variants among the genetic signatures of patients. Deleterious variants can be determined using, for example, the SnpEff and Variant Effect Predictor (www.ensembl.org) engines. SnpEff is capable of rapidly categorizing the effects of SNPs and other variants in whole genome sequences. See, Cingolani et al., A program for annotating and predicting the effects of single nucleotide polymorphisms, SnpEff: SNPs in the genome of Drosophila melanogaster strain w¹¹¹⁸; iso-2; iso-3; Landes Bioscience, 6:2, 1-13; April/May/June 2012, incorporated herein by reference. Variants predicted to have a high impact or be “moderate missense variants” (moderate is defined by SnpEff as causing an amino acid change) using programs such as SnpEff are then selected.

Upon identification of these high and moderate impact variants, the variants are then passed through a scoring system based on various annotation tools. One of ordinary skill in the art would understand that both molecular and computational approaches are available for annotating variants (e.g., by comparing to a known database, through the use of ANOVA technology, through the use of multivariant analysis). Exemplary annotation tools include the Database for Annotation, Visualization and Integrated Discover (DAVID). Nature Protocols 2009; 4(1):44; and Nucleic Acids Res. 2009; 37(1):1, incorporated herein by reference.

Variants that were considered deleterious by at least two annotation tools can then be passed through to the association analysis, along with the microbiome and clinical data to determine whether the genetic variant signatures obtained from the subjects are associated with their potential for reproductive success.

The association analysis involves the use of any one of a number of models to calculate the potential for reproductive success for the reference population, such as a cohort of patients, as described above with respect to the “Analysis of Microorganisms” section.

One method for determining the effect that genetic information has on the potential for reproductive success includes the sequence kernel association testing (SKAT) method, which is a gene set level methodology for testing if SNP-sets (gene sets) are associated with phenotypes (continuous or discrete) of interest. See Wu M C, Lee S, Cai T, Li Y, Boehnke M, Lin X. Rare-Variant Association Testing for Sequencing Data with the Sequence Kernel Association Test. American Journal of Human Genetics. 2011; 89(1):82-93. doi:10.1016/j.ajhg.2011.05.029, incorporated herein by reference. For additional description of the incorporation of genetic factors into a reproductive fertility model, and specifically regarding the use of SKAT in adjusting the model, see U.S. Provisional Application No. 62/408,632, filed Oct. 14, 2016, incorporated herein by reference. Furthermore, burden testing can be used to enhance the results of the SKAT analysis given that SKAT only provides a P-value for evidence of an association between the SNP-set and phenotype of interest. Adjustment of models using SKAT-type analysis, allows one to see whether there is statistical evidence that genomic information, at the category level (e.g., functional biological classification level), provides additional information beyond known microbiological and clinical metrics that is sufficient to significantly affect the model, and therefore be associated with the potential for reproductive success.

Once the model has been developed based on a reference set of data, as described above with respect to the analysis of microorganisms, the model can be applied to data obtained from an individual, or patient, in order to predict the potential for reproductive success.

Methods for Recommending Treatment and/or Treating a Patient

In certain embodiment, methods include recommending and/or prescribing a fertility-related treatment. The recommended/prescribed treatment protocol will depend, in part, on the potential generated in accordance with the description above. Methods of the invention can also involve the generation of a report which includes the individual's potential for reproductive success, and optionally, a recommended treatment protocol.

Exemplary fertility treatments include, but are not limited to, assisted reproductive technologies (ART), non-ART fertility treatments (RE), and fertility preservation technologies (egg, embryo, or ovarian preservation). Exemplary assisted reproductive technologies include, without limitation, in vitro fertilization (IVF), zygote intrafallopian transfer (ZIFT), gametic intrafallopian transfer (GIFT), or intracytoplasmic sperm injection (ICSI) paired with one of the methods above.

In IVF, eggs are removed from the female subject, fertilized outside the body, and implanted inside the uterus of the female subject. ZIFT is similar to IVF in that eggs are removed and fertilization of the eggs occurs outside the body. In ZIFT, however, the eggs are implanted in the Fallopian tube rather than the uterus. GIFT involves transferring eggs and sperm into the female subject's Fallopian tube. Accordingly, fertilization occurs inside the woman's body. In ICSI, a single sperm is injected into a mature egg that has removed from the body. The embryo is then transferred to the uterus or Fallopian tube. In RE, hormone stimulation is used to improve the woman's fertility. Exemplary fertility preservation treatments include egg freezing in which eggs are removed, vitrified or otherwise frozen, and then stored indefinitely. Preservation can similarly be achieved through cryo-preservation of embryos generated through IVF and cryo-preservation of ovarian tissue, including slices of the ovarian cortex. Preservation could also involve removal of the ovary from the pelvic region and subcutaneous implantation in an ectopic location such as under the skin the in periphery of the body (i.e., arm).

Exemplary non-ART fertility treatments include ovulation induction protocols with or without intrauterine insemination (IUI) with sperm. Exemplary ovulation induction agents include gonadotropins such as luteinizing hormone (LH), follicle stimulating hormone (FSH), and human chorionic gonadotropin (hCG); and oral ovulation induction agents such as letrozole, clomiphene citrate, bromocriptine, metformin, and cabergoline.

### Systems

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location, such as in a reference database, and the computer communicates across a network to access the reference set to compare data derived from the individual to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Python, R, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

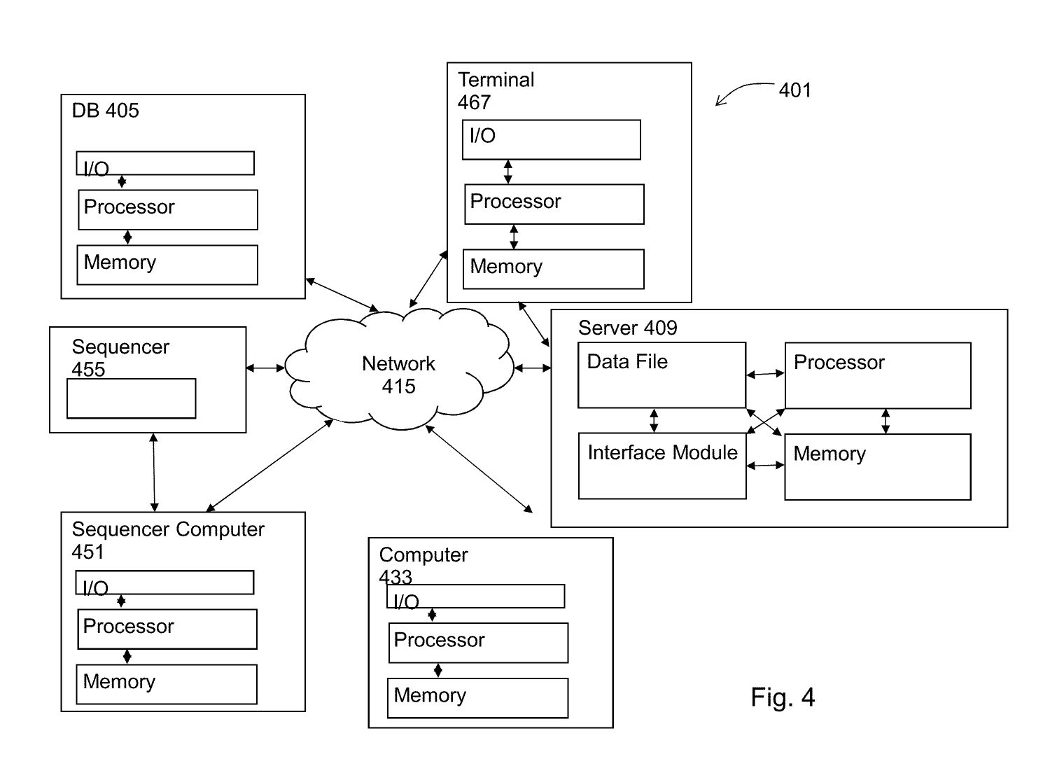

In an exemplary embodiment shown in FIG. 4, system 401 can include a computer 433 (e.g., laptop, desktop, or tablet). The computer 433 may be configured to communicate across a network 415. Computer 433 includes one or more processor and memory as well as an input/output mechanism. Where methods of the invention employ a client/server architecture, any steps of methods of the invention may be performed using server 409, which includes one or more of processor and memory, capable of obtaining data, instructions, etc., or providing results via interface module or providing results as a file. Server 409 may be engaged over network 415 through computer 433 or terminal 467, or server 415 may be directly connected to terminal 467, including one or more processor and memory, as well as input/output mechanism. In some embodiments, systems include an instrument 455 for obtaining sequencing data, antibody-based detection data, and/or PCR data, which may be coupled to a computer 451 for initial processing of sequence reads, PCR data, and detection data.

Memory according to the invention can include a machine-readable medium on which is stored one or more sets of instructions (e.g., software) embodying any one or more of the methodologies or functions described herein for generating an individual's potential for reproductive success. The software may also reside, completely or at least partially, within the main memory and/or within the processor during execution thereof by the computer system, the main memory and the processor also constituting machine-readable media. The software may further be transmitted or received over a network via the network interface device.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations.

### Examples

In this study, three saliva samples were collected from subjects using a saliva collection kit. Sequencing of the DNA was carried out on Illumina HiSeq-II sequencing machines using a paired-end sequencing library preparation protocol. The output reads were then mapped to the human genome reference sequence (hg19) using BWA. All read sequences that did not map to the human genome were retained and then remapped to the HOMD oral microbiome reference genome (i.e., around 1.3 giga-basepairs of DNA comprising 461 oral microbiome species). Some species were incomplete genomes, meaning the contiguous sequences or scaffolds which comprised their genetic material had to be merged to form a whole genome.

The full length of each of the 461 species was then calculated, this genomic length (together with the full count of reads mapped along the full length of the genome) being required to calculate the normalized abundance per species, per sample. Only those reads which were deemed properly paired at the alignment stage were used to calculate species abundance. All other reads were filtered out to ensure no singletons, misaligned, or cross chromosomal reads were included in the analysis. Tables 4 through 7 summarize these calculations.

TABLE 4
Normalized Abundances of Species in all Samples, Ordered by Average Sample Abundance

| | Genome | | | |
|---|---|---|---|---|
| Species Name and Reference Number | Length (bp) | Sample 1 | Sample 2 | Sample 3 |
| Prevotella melaninogenica ATCC 25845 | 3168282 | 129726.401 | 241548.67 | 752888.13 |
| Porphyromonas sp. OT 278 W7784 | 2146981 | 564890.858 | 45126.47 | 18257.15 |
| Prevotella pallens ATCC 700821 | 3043692 | 113184.759 | 477258.39 | 18090.4 |
| Prevotella melaninogenica D18 | 3212205 | 71907.351 | 150822.05 | 369159.32 |
| Prevotella sp. oral taxon 306 F0472 | 2945767 | 79147.001 | 239075.38 | 23116.29 |
| Prevotella salivae DSM 15606 | 3140543 | 33493.223 | 115195.31 | 170416.95 |
| Veillonella atypica ACS-134-V-Col7a | 2151913 | 51643.867 | 150269.99 | 108586.84 |
| Actinomyces sp. oral taxon 172 F0311 | 2459518 | 136933.383 | 18840.89 | 55742.72 |
| Veillonella dispar ATCC 17748 | 2116567 | 26083.909 | 76227.22 | 103567.97 |
| Actinomyces odontolyticus ATCC 17982, DSM 43331 | 2393758 | 46213.711 | 10753.62 | 110298.42 |
| Veillonella sp. oral taxon 158 F0412 | 2176752 | 90304.805 | 34492.34 | 29646.61 |
| Prevotella scopus JCM 17725 | 3184425 | 21446.896 | 49066.96 | 73103.38 |
| Haemophilus parainfluenzae ATCC 33392 | 2109295 | 94875.005 | 10078.51 | 25319.01 |
| Haemophilus parainfluenzae T3T1 | 2086875 | 87883.858 | 14339.18 | 25231.37 |
| Prevotella histicola JCM 15637 = DNF00424 | 2949807 | 6622.346 | 41160.13 | 64889.2 |

TABLE 5
Five Most Abundant Species Found in Sample 1

| | Genomic | Normalized Abundance | | |
|---|---|---|---|---|
| Species and Reference Number | Length (bp) | Sample 1 | Sample 2 | Sample 3 |
| Porphyromonas sp. OT 278 W7784 | 2346981 | 564890.86 | 45126.47 | 18257.15 |
| Actinomyces sp. oral taxon 172 F0311 | 2459538 | 136933.38 | 18840.89 | 55742.72 |
| Prevotella melaninogenica ATCC 25845 | 3368282 | 329726.4 | 241548.67 | 752888.13 |
| Prevotella pallens ATCC 700821 | 3043692 | 113184.76 | 477258.39 | 18090.4 |
| Haemophilus parainfluenzae ATCC 33392 | 2109295 | 94875.01 | 10078.51 | 25319.01 |

TABLE 6
Five Most Abundant Species Found in Sample 2

| | Genomic | Normalized Abundance | | |
|---|---|---|---|---|
| Species and Reference Number | Length (bp) | Sample 1 | Sample 2 | Sample 3 |
| Prevotella pallens ATCC 700821 | 3043692 | 113184.76 | 477258.4 | 38090.4 |
| Prevotella melaninogenica ATCC 25845 | 3168282 | 129726.4 | 241548.7 | 752888.33 |
| Prevotella sp. oral taxon 306 F0472 | 2945767 | 79147 | 239075.4 | 23116.29 |
| Prevotella melaninogenica D18 | 3212205 | 71907.35 | 150822 | 369159.32 |
| Veillonella atypica ACS-134-V-Col7a | 2151913 | 51643.87 | 350270 | 108586.84 |

TABLE 7
Five Most Abundant Species Found in Sample 3

| | Genomic | Normalized Abundance | | |
|---|---|---|---|---|
| Species and Reference Number | Length (bp) | Sample 1 | Sample 2 | Sample 3 |
| Prevotella melaninogenica ATCC 25845 | 3168282 | 129726.4 | 241548.67 | 752888.1 |
| Prevotella melaninogenica D18 | 3212205 | 71907.35 | 150822.05 | 369159.3 |
| Prevotella salivae DSM 15606 | 3140543 | 33493.22 | 115195.31 | 370436.9 |
| Actinomyces odontolyticus ATCC 17982, DSM 43331 | 2393758 | 46233.71 | 10753.62 | 310298.4 |
| Veillonella atypica ACS-134-V-Col7a | 2151913 | 51643.87 | 150269.99 | 308586.8 |

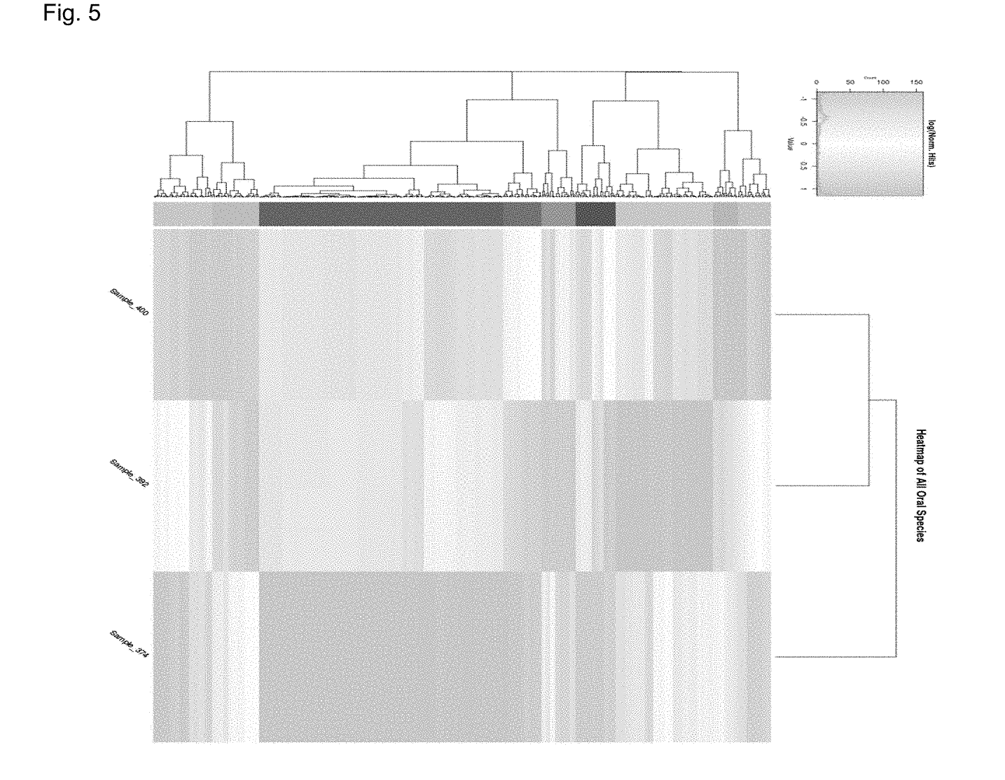

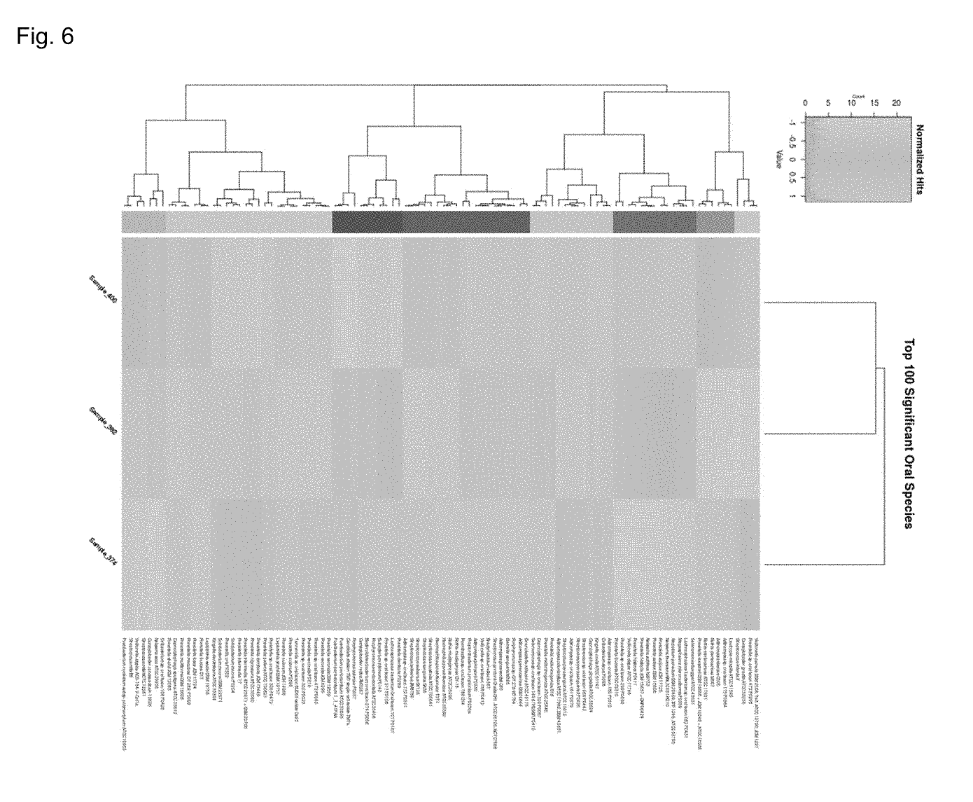

A matrix of normalized abundance rates for all species and the 100 most abundant species was generated and used to plot a clustered heatmap (columns are samples and the rows are species) as shown in FIG. 5 and FIG. 6, respectively.

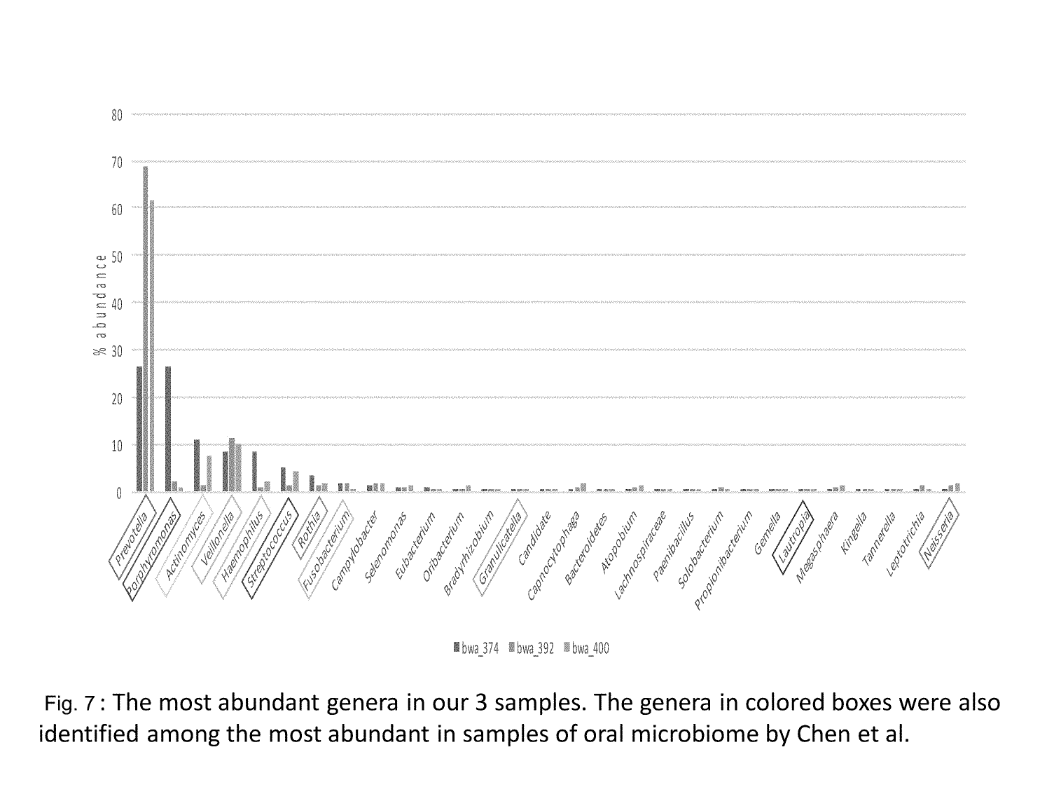

When we compared the annotated oral species for which there were complete genome sequences to those that were identified in our reported full-genome species, we verified that complete capture was achieved. We observed that the capture levels across all samples differ, indicating that the microbiome structure uniquely differs among individuals. FIG. 7 depicts the different species clusters identified in each sample.

To confirm that the findings are consistent with what is known about the oral microbiome, we compared the most abundant genera in the samples (FIG. 7) to the ten (10) most abundant genera identified in previously-published reports: Streptococcus, Prevotella, Neisseria, Haemophilus, Porphyromonas, Gemella, Rothia, Granulicatella, Fusobacterium, Actinomyces, and Veillonella (Chen H, Jiang W. Application of high-throughput sequencing in understanding human oral microbiome related with health and disease. Frontiers in Microbiology. 2014; 5:508. doi:10.3389/fmicb.2014.00508). These genera were also identified by our analysis and eight (Prevotella, Porphyromonas, Actinomyces, Veillonella, Haemophilus, Streptococcus, Rothia, and Fusobacterium) were also identified to be the most abundant genera in our samples. This analysis demonstrates that our methodologies produced results consistent with what is known in the literature.

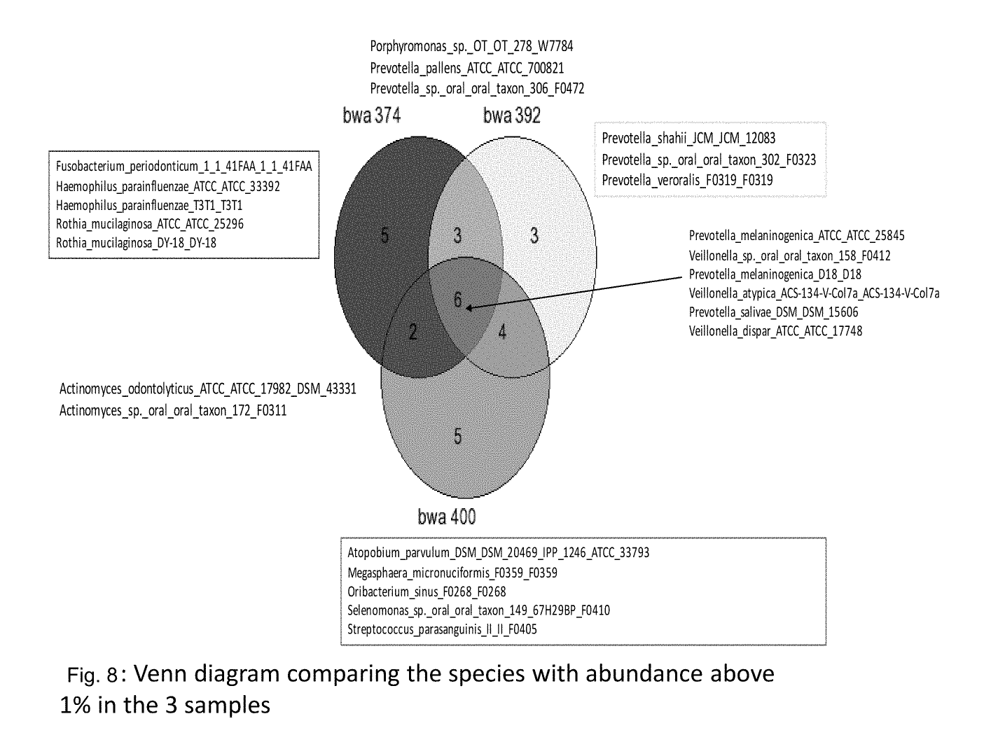

We then identified the most abundant species in each sample by calculating the relative abundance of each species in each sample, and then compared each species with an abundance above 1% across the three samples (FIG. 8).

We then analyzed the microbiome profile of each sample in light of their clinical information and reproductive phenotypes, specifically analyzing the hormonal levels and reproductive conditions (Table 8).

TABLE 8
Sample Demographics

| | | Baseline | Baseline | Baseline | First | First | | |
|---|---|---|---|---|---|---|---|---|
| | | FSH | LH | E2 | AMH | TSH | First | |
| | BMI | (mIU/mL) | (IU/L) | (pg/mL) | (ng/mL) | (ng/mL) | BAFC | Clinical Diagnosis |
| Sample 1 | 20.9 | 8.0 | 2.2 | 94.2 | 0.5 | 1.7 | 6 | Diminished Ovarian |
| | | | | | | | | Reserve and Recurrent |
| | | | | | | | | Pregnancy Loss |
| Sample 2 | 24.5 | 3.8 | 4.2 | 54.7 | — | — | 13 | Idiopathic Infertility |
| Sample 3 | 25.2 | 6.3 | 7.1 | 38.1 | 1.7 | 4.1 | 13 | Uterine factor and |
| | | | | | | | | Idiopathic Infertility |

We identified that Sample 1 had the most negative reproductive parameters typical of ovarian dysfunction and poor oocyte quality (lowest AMH and highest FSH). Sample 1 had a microbiome profile containing increased levels of Haemophilus parainfluenzae and Rothia mucilaginosa whereas these species are absent or present at low abundance in the other samples analyzed. In sum, a microbiome profile of a woman with an increased relative abundance of Haemophilus parainfluenzae and Rothia mucilaginosa correlates with a negative reproductive outcome, specifically with Diminished Ovarian Reserve (DOR) and Recurrent Pregnancy Loss (RPL).

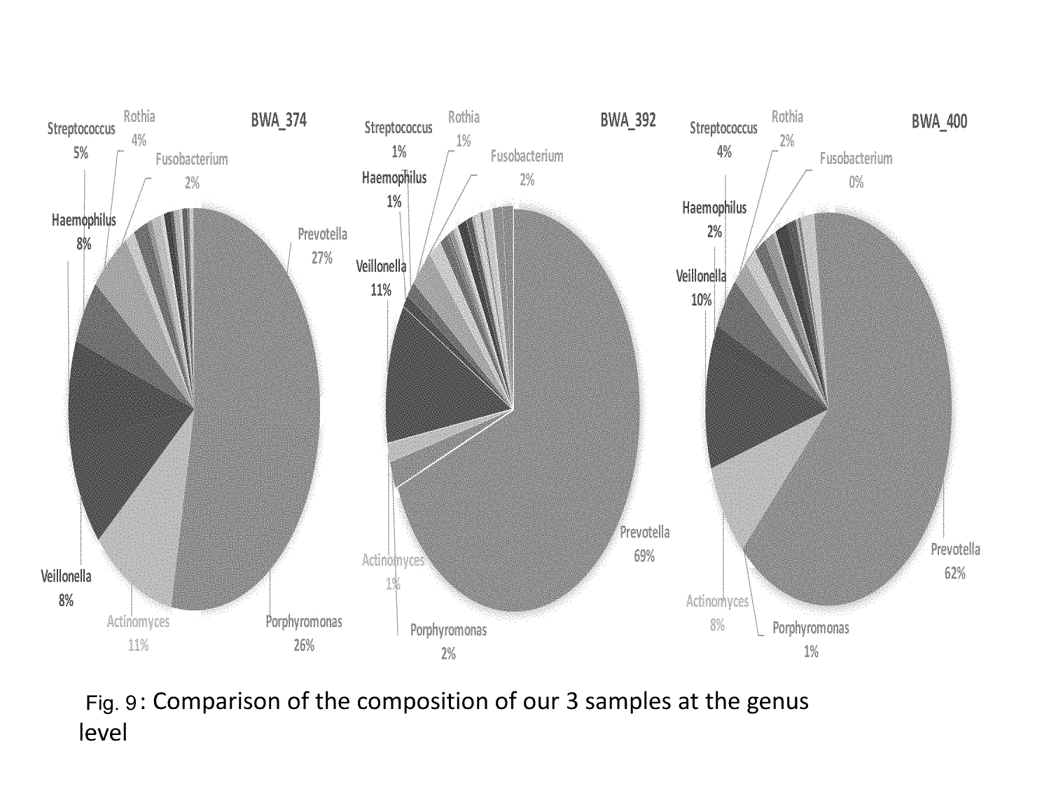

We also compared the overall composition of the samples by identifying the most abundant genera and their relative abundance in each sample. We observed that the samples from women diagnosed with Idiopathic Infertility (Samples 2 and 3) have a relative abundance of 60-70% Prevotella and 1-2% of Porphyromonas. Whereas, Sample 1 has lower abundance of Prevotella and a greater relative abundance of Porphyromonas (FIG. 9). This analysis shows that there is an association between the overall degree of diversity of the sample or the proportion of the abundance of specific genera and reproductive phenotypes. Specifically, an increased relative abundance of Porphyromonas is associated with negative reproductive outcomes.

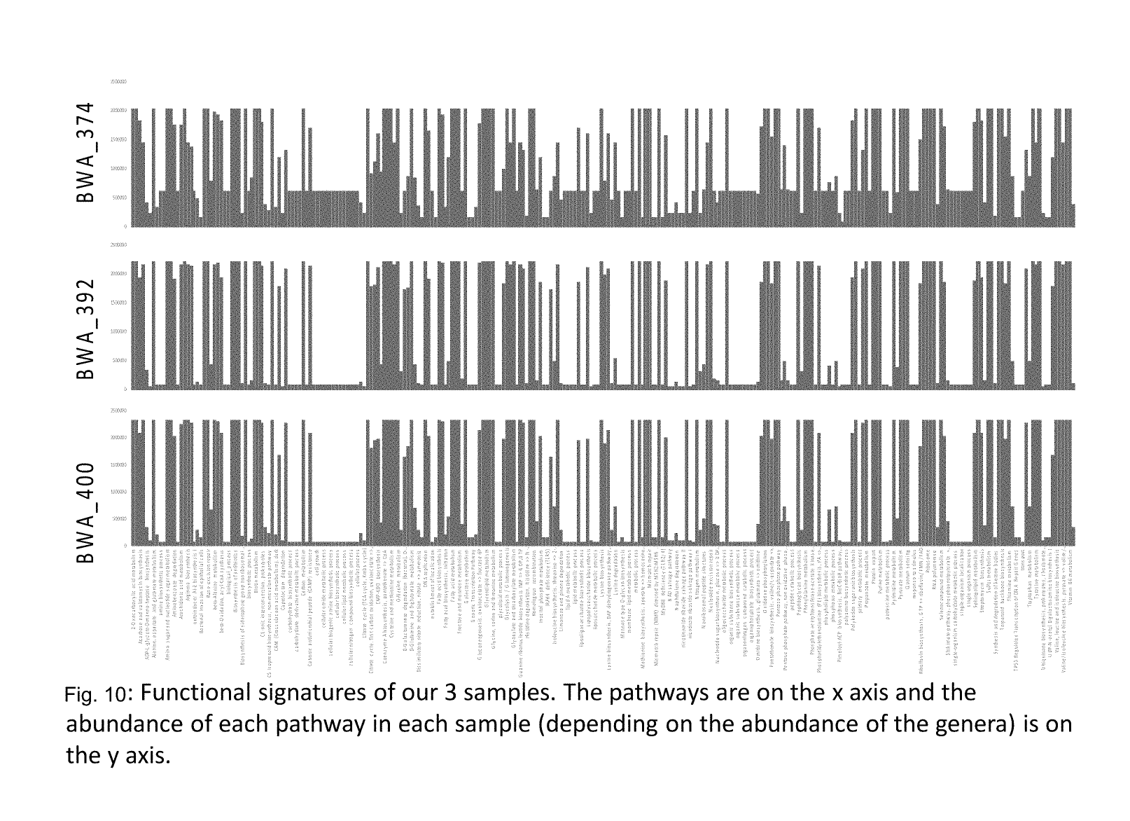

To test how the 3 samples differ at a functional level, we generated functional signatures of each sample by identifying all the biological processes described as being associated with each genus present in the 3 samples (source: https://www.ncbi.nlm.nih.gov/biosystems/). We generated a “functional signature” of each sample by combining the biological processes specific for each genus with the abundance of each genus in a sample (FIG. 10). We observed that the 3 samples have different functional signatures corresponding to a difference in the biological processes carried out by the microorganisms in each sample. In particular, the patient diagnosed with DOR and RPL has a higher abundance of a specific set of biological processes compared to the two samples from patients diagnosed with idiopathic infertility.

We identified species or genera associated with positive or negative reproductive outcomes by reviewing the published literature and compiling lists of species or genera associated with negative, neutral, or positive reproductive outcomes (Table 9).

TABLE 9
Studies Identifying Species or Genera Associated with Negative, Neutral,
or Positive Reproductive Outcomes (Each study is identified by its PMID.)

| REPRODUCTIVE | | | |
|---|---|---|---|
| OUTCOME | | | |
| (reproductive aspect) | MICROORGANISMS | CORRELATION | PMID |
| Positive (Preterm | Prevotella nigrescens, | Significantly decreased risk of | 15691348 |
| Birth (PTB)) | Aggregatibacter actinomycetemcomitans | preterm delivery of low birth | |
| | | weight babies | |
| Positive (PTB) | Paenibacillus spp. | Enriched in term placental | 24848255 |
| | | specimens | |
| Positive (PTB) | Lactobacillus spp. | Absence of lactobacilli | 12530101 |
| | | (sensitivity (28%) and positive | |
| | | predictive value (25%)) was a | |
| | | predictor of preterm delivery | |
| | | at <33 weeks of gestation | |
| Positive (PTB) | Lactobacillus crispatus | Low median levels | 18999913 |
| | | of Lactobacillus crispatus were | |
| | | significantly predictive of PTB | |
| Positive (None, | Lactobacillus crispatus, Lactobacillus gasseri, | Healthy vaginal communities are | 20534435 |
| Overall vaginal | Lactobacillus iners, Lactobacillus jensenii | typically dominated by only one | |
| health) | | or two of these species | |
| Positive | Lactobacillus crispatus | Colonizing the transfer-catheter | 24390919 |
| (Implantation and | | tip with Lactobacillus crispatus | |
| Live Birth) | | at the time of embryo transfer | |
| | | may increase the rates of | |
| | | implantation and live birth rate | |
| | | while decreasing the rate of | |
| | | infection | |
| Neutral | Actinobacteria spp. | Patients with PCOS showed a | 27610099 |
| (Polycystic | | reduced salivary relative | |
| Ovarian Syndrome | | abundance of Actinobacteria | |
| (PCOS)) | | | |
| Neutral (None) | Firmicutes spp., Tenericutes spp., | Most common species found in | 24848255 |
| | Proteobacteria spp., Bacteroides spp., | human placenta | |
| | and Fusobacteria spp. | | |
| Negative (PTB) | Porphyromonas gingivalis, Tannerella forsythia, | Bacterial organisms significantly | 17470016 |
| | Treponema denticola, Prevotella intermedia, | associated with periodontal | |
| | Prevotella nigrescens, Campylobacter rectus | disease were also associated with | |
| | | PTB, albeit at borderline | |
| | | significance (p = 0.012-0.069) | |
| Negative (PTB) | Mycoplasma hominis | Presence of Mycoplasma hominis | 12530101 |
| | | (sensitivity (7%) and positive | |
| | | predictive value (13%)) was a | |
| | | predictor of preterm delivery | |
| | | at <33 weeks of gestation | |
| Negative (PTB) | Peptostreptococcus micros and | Significantly increased risk of | 15691348 |
| | Campylobacter rectus | preterm delivery of low birth | |
| | | weight babies | |
| Negative (PTB) | Ureaplasma urealyticum, Mycoplasma hominis, | Organisms commonly cultured | 16953371 |
| | Bacteroides spp., Gardnerella vaginalis, | from the amniotic cavity | |
| | Neisseria gonorrhoeae, Chlamydia trachomatis, | following preterm delivery | |
| | Trichomonas vaginalis, and Streptococcus agalactiae | | |
| Negative (PTB) | Burkholderia spp. | Preterm placentas had changes in | 24848255 |
| | | abundance | |
| Negative (PTB) | Bergeyella spp. | Same strain identified in oral | 16597879 |
| | | cavity and amniotic fluid (not in | |
| | | the vagina) of PTB patient | |
| Negative (PTB) | Capnocytophaga spp. | Isolated in amniotic fluid during | 4061534, |
| | | preterm labor | 10221619, |
| | | | 10458530 |
| Negative (PTB) | Ureaplasma parvum, Ureaplasma urealyticum, | Most commonly associated | 25505898 |
| | Mycoplasma hominis, Gardnerella vaginalis, | organisms with AF infection and | |
| | Peptostreptococcus spp., Enterococcus spp., | PTB | |
| | Streptococcus spp. (particularly S. agalactiae), | | |
| | Fusobacterium nucleatum, Leptotrichia spp., | | |
| | Sneathia sanguinegens, Haemophilus influenzae, | | |
| | Escherichia coli | | |
| Negative (PTB) | Porphyromonas gingivalis | Dental Infection of | 26322971 |
| | | Porphyromonas gingivalis | |
| | | induces preterm birth in mice | |
| Negative (PTB) | Ureaplasma urealyticum | Ureaplasmal infection of the | 8457981 |
| | | chorioamnion is significantly | |
| | | associated with premature | |
| | | spontaneous labor and delivery | |
| Negative (PTB) | Gardnerella vaginalis | High median levels | 18999913 |
| | | of Gardnerella vaginalis were | |
| | | significantly predictive of SPTB | |
| Negative (Pre- | Aggregatibacter actinomycetcmcomitans | Levels of maternal subgingival | 22393563 |
| eclampsia) | | A. actinomycetemcomitans DNA | |
| | | were elevated in preeclamptic | |
| | | women. | |
| Negative (Pre- | Porphyromonas gingivalis, Tannerella forsythia, and | Chronic periodontal disease and | 16460242 |
| eclampsia) | Eikenella corrodens | the presence of P. gingivalis, | |
| | | T. forsythensis, and E. corrodens | |
| | | were significantly associated | |
| | | with preeclampsia in pregnant | |
| | | women | |
| Negative (PCOS) | Porphyromonas gingivalis, Fusobacterium nucleatum, | Higher level in women | 25232962 |
| | Streptococcus oralis, Tannerella forsythia | diagnosed with PCOS compared | |
| | | to healthy women | |

We consolidated this data and compiled a list of species associated with negative and positive reproductive outcomes:
POSITIVE: Prevotella nigrescens, Aggregatibacter actinomycetemcomitans, Lactobacillus crispatus, Lactobacillus gasseri, Lactobacillus iners, and Lactobacillus jensenii
NEGATIVE: Aggregatibacter actinomycetemcomitans, Campylobacter rectus, Chlamydia trachomatis, Eikenella corrodens, Escherichia coli, Fusobacterium nucleatum, Gardnerella vaginalis, Haemophilus influenza, Mycoplasma hominis, Neisseria gonorrhoeae, Porphyromonas gingivalis, Prevotella intermedia, Prevotella nigrescens, Sneathia sanguinegens, Tannerella denticola, Tannerella forsythia, Trichomonas vaginalis, Ureaplasma parvum, Ureaplasma urealyticum, and Porphyromonas gingivalis

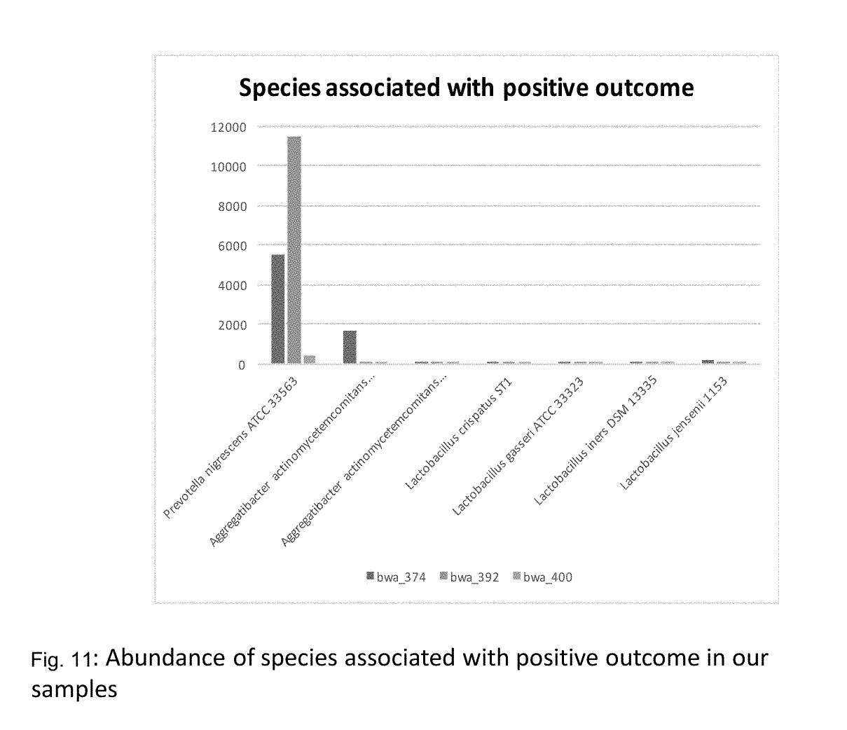

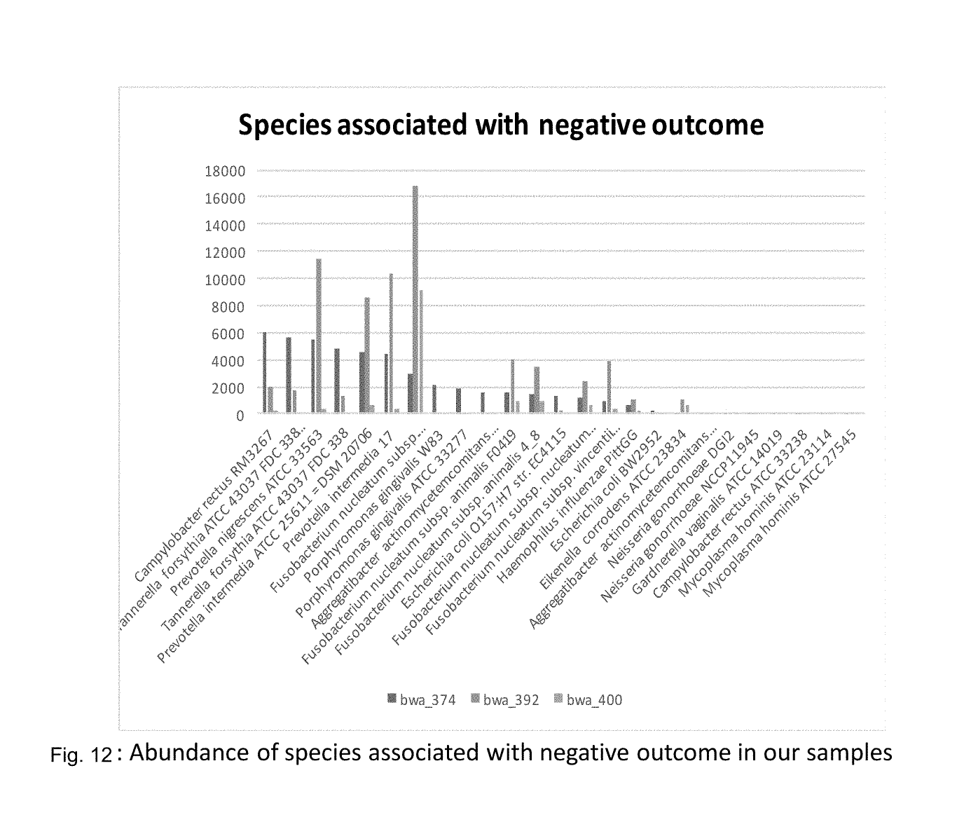

We identified the abundance of these genera and species in our samples and observed that our 3 samples show different abundance of species associated with negative and positive reproductive outcomes (FIG. 11 and FIG. 12). In particular, the sample from the patient diagnosed with uterine factor/idiopathic infertility (Sample 3) shows the lowest abundance of some of the species associated with positive reproductive outcome, while each one of the 3 samples show a higher abundance of a sub-set of the species associated with negative reproductive outcomes.

The differences between samples with different phenotypes suggest that there is an association between high or low abundance of certain species and specific positive or negative reproductive outcomes.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore included.

What is claimed is:

1. A method for the assessment of potential reproductive success, the method comprising the steps of
obtaining a body fluid sample from a patient;
conducting an assay to identify a plurality of microorganisms present in said sample,
processing said plurality of microorganisms in order to obtain a subset of the microorganisms;
comparing the subset to a reference set of microorganisms known to be associated with reproductive success; and
informing said patient of potential reproductive success based upon a statistically-significant match between the subset and the reference set.

2. The method of claim 1, wherein the body fluid is selected from a vaginal secretion, an anal secretion, an oral secretion, and a nasal secretion.

3. The method of claim 2, wherein the oral secretion is saliva.

4. The method of claim 1, wherein the microorganisms are selected from bacteria, virus, and eukaryotic microorganisms.

5. The method of claim 1, wherein the processing step comprises identifying microorganisms in the sample and sorting the microorganisms by genus and/or species.

6. The method of claim 5, further comprising selecting microorganisms suspected to influence reproductive outcome.

7. The method of claim 1, wherein the conducting step comprises sequencing nucleic acids of the microorganisms.

8. The method of claim 1, wherein the conducting step comprises antibody-based detection of the microorganisms.

9. The method of claim 1, wherein one or more microorganisms in the subset are selected from the group consisting of Abiotrophia spp., Achromobacter spp., Acinetobacter spp., Actinobaculum spp., Actinomyces spp., Afipia spp., Aggregatibacter spp., Agrobacterium spp., Alloiococcus spp., Alloscardovia spp., Anaerococcus spp., Anaeroglobus spp., Arcanobacterium spp., Atopobium spp., Bacillus spp., Bacteroides spp., Bacteroidetes spp., Bartonella spp., Bifidobacterium spp., Bordetella spp., Bradyrhizobium spp., Brevundimonas spp., Bulleidia spp., Burkholderia spp., Campylobacter spp., Candida spp., Capnocytophaga spp., Cardiobacterium spp., Catonella spp., Centipeda spp., Chlamydophila spp., Chloroflexi spp., Clostridiales spp., Comamonas spp., Corynebacterium spp., Cronobacter spp., Cryptobacterium spp., Delftia spp., Desulfobulbus spp., Dialister spp., Dolosigranulum spp., Eggerthella spp., Eikenella spp., Enterobacter spp., Enterococcus spp., Erysipelothrix spp., Escherichia spp., Eubacterium spp., Filifactor spp., Finegoldia spp., Fusobacterium spp., Gardnerella spp., Gemella spp., Granulicatella spp., Haemophilus spp., Helicobacter spp., Johnsonella spp., Jonquetella spp., Kingella spp., Klebsiella spp., Kytococcus spp., Lachnospiraceae spp., Lactobacillus spp., Lactococcus spp., Lautropia spp., Leptotrichia spp., Listeria spp., Lysinibacillus spp., Megasphaera spp., Mesorhizobium spp., Methanobrevibacter spp., Microbacterium spp., Mitsuokella spp., Mobiluncus spp., Mogibacterium spp., Moraxella spp., Mycobacterium spp., Mycoplasma spp., Neisseria spp., Ochrobactrum spp., Olsenella spp., Oribacterium spp., Paenibacillus spp., Parascardovia spp., Parvimonas spp., Peptoniphilus spp., Peptostreptococcacea spp., Peptostreptococcus spp., Porphyromonas spp., Prevotella spp., Propionibacterium spp., Proteus spp., Pseudomonas spp., Pseudoramibacter spp., Pyramidobacter spp., Ralstonia spp., Rhodobacter spp., Rothia spp., Sanguibacter spp., Scardovia spp., Selenomonas spp., Shuttleworthia spp., Simonsiella spp., Slackia spp., Solobacterium spp., Staphylococcus spp., Stenotrophomonas spp., Streptococcus spp., Synergistetes spp., Tannerella spp., Treponema spp., Turicella spp., Variovorax spp., Veillonella spp., and Yersinia spp.

10. The method of claim 1, further comprising prescribing a course of treatment.

11. The method of claim 10, wherein the course of treatment is selected from the group consisting of assisted reproductive technologies (ART), non-ART fertility treatments (RE), and fertility preservation technologies.

12. The method of claim 1, wherein said comparing step comprises referencing a population of microorganisms known or suspected to affect reproductive outcomes.

13. The method of claim 12, wherein said population comprises a set of microorganisms associated with reproductive success.

14. The method of claim 13, wherein said set comprises Prevotella nigrescens, Aggregatibacter actinomycetemcomitans, Paenibacillus spp., Lactobacillus crispatus, Lactobacillus gasseri, Lactobacillus iners, and Lactobacillus jensenii.

15. The method of claim 1, further comprising determining an amount of one or more microorganisms in the subset of microorganisms.

16. The method of claim 15, further comprising comparing the amount of one or more microorganisms in the subset to amounts microorganisms in the reference set.

17. The method of claim 1, further comprising obtaining clinical data from the patient.

18. The method of claim 17, further comprising analyzing the clinical data from the patient against data from a reference population.

19. The method of claim 1, further comprising obtaining genetic data from the patient.

20. The method of claim 19, further comprising analyzing the genetic data from the patient against data from a reference population.

21. A method for analyzing reproductive success of an individual, the method comprising:
obtaining a body fluid sample from a patient;
conducting an assay on the sample to determine a quantity of microorganisms present in the sample;
comparing the quantity to a reference set of data; and
informing said patient of potential reproductive success based upon the comparison.

22. A method for analyzing reproductive success of an individual, the method comprising:
obtaining a body fluid sample from an individual;
conducting an assay on the sample determine a diversity of microorganisms within the individual;
comparing the diversity of the individual to a reference set of data; and
informing said patient of potential reproductive success based upon the comparison.
